(19) United States
(12) Patent Application Publication
(10) Pub. No.: US 2024/0301092 A1
(43) Pub. Date: Sep. 12, 2024

(54) HEPARIN FROM MODIFIED MST CELLS AND METHODS OF MAKING AND USING

(71) Applicant: TEGA Therapeutics, Inc., San Diego, CA (US)

(72) Inventors: Bryan E. THACKER, San Diego, CA (US); Jeffrey D. ESKO, San Diego, CA (US); Charles A. GLASS, San Diego, CA (US)

(21) Appl. No.: 18/575,016

(22) PCT Filed: Jun. 29, 2022
(86) PCT No.: PCT/US2022/035599

§ 371 Date: Dec. 28, 2023

Related U.S. Application Data
(60) Provisional application No. 63/216,495, filed on Jun. 29, 2021.

(51) Int. Cl. C08B 37/00; A61K 31/727; C08L 5/10; C12N 5/00; C12N 5/0787; C12N 9/10

(52) U.S. Cl. CPC C08B 37/0075; A61K 31/727; C08L 5/10; C12N 5/0018; C12N 5/0642; C12N 9/13; C12N 2500/34; C12N 2510/02; C12N 2511/00

(57) ABSTRACT

Provided herein are methods of producing heparin and heparan sulfate from modified cells, such as modified MST cells, and compositions comprising heparin and heparan sulfate isolated from modified cells.

### CROSS-REFERENCE

This application claims the benefit of U.S. Provisional Application No. 63/216,495, filed Jun. 29, 2021, which is incorporated herein by reference in its entirety.

### STATEMENT AS TO FEDERALLY SPONSORED RESEARCH

This invention was made with government support under 1622959, 1842736, and 2026188 awarded by the National Science Foundation. The government has certain rights in the invention.

### BACKGROUND

Heparin remains an important and widely prescribed drug. Approximately 300,000 doses are administered per day in the United States. An aging population and increased incidence of diseases (e.g. heart failure and diabetes) leads to more procedures like cardiopulmonary bypass surgery where heparin remains the drug of choice. Risks of heparin induced thrombocytopenia (HIT) associated with high doses of heparin administered before and during surgery have led to the introduction of alternative anticoagulant therapies.

### SUMMARY

In an aspect, provided herein are methods of producing a heparin or heparan sulfate. In some cases, the method comprises culturing a genetically modified mastocytoma cell line; and isolating the heparin or heparan sulfate from the cell line, wherein the genetically modified cell line overexpresses one or more of heparan sulfate-glucosamine 3-sulfotransferae 1 (Hs3st1), heparan sulfate 6-O-sulfotransferase 1 (Hs6st1), heparan sulfate 6-O-sulfotransferase 2 (Hs6st2), and N-deacetylase and N-sulfotransferase 2 (Ndst2). In some cases, the genetically modified cell line is deficient for one or more of chondroitin sulfate N-acetylgalactosaminyltransferase 1 (Csgalnact1), chondroitin sulfate N-acetylgalactosaminyltransferase 2 (Csgalnact2), and chondroitin sulfate synthase 1 (Chsy1). In some cases, the mastocytoma cell line is selected from the group consisting of MST cells, P815 cells, MC/9 cells, SI/SI4 cells, 10P2 cells, 11P0-1 cells, and 10P12 cells. In some cases, the genetically modified cell line is deficient for a heparan sulfate catabolic enzyme. In some cases, the method comprises degranulating the cell line. In some cases, degranulating the cell line comprises a method selected from the group consisting of Antigen-IgE induced FcεRI aggregation on the degranulating cell surface, contacting the cell line to a degranulating agent, altering the culture temperature, altering the culture medium pH, altering the culture medium salt concentration, and agitation. In some cases, the degranulating agent is selected from the group consisting of calcium ionophore A23187, compound 48/80, tetradecanoyl phorbol acetate (TPA), and substance P. In some cases, the genetically modified cell line is cultured in CDM4NS0 medium. In some cases, the genetically modified cell line is cultured in a medium supplemented with xylosides. In some cases, the genetically modified cell line is cultured in suspension culture or in a hollow fiber bioreactor.

In another aspect, there are provided methods of reducing blood clots in a subject. In some cases, the method comprising administering a composition comprising a heparin or a heparan sulfate produced according to any method provided herein.

In another aspect, there are provided, cells comprising a genetically modified mastocytoma cell that overexpresses one or more of heparan sulfate-glucosamine 3-sulfotransferae 1 (Hs3st1), heparan sulfate 6-O-sulfotransferase 1 (Hs6st1), heparan sulfate 6-O-sulfotransferase 2 (Hs6st2), and N-deacetylase and N-sulfotransferase 2 (Ndst2). In some cases, the cell is deficient for one or more of chondroitin sulfate N-acetylgalactosaminyltransferase 1 (Csgalnact1), chondroitin sulfate N-acetylgalactosaminyltransferase 2 (Csgalnact2), and chondroitin sulfate synthase 1 (Chsy1). In some cases, the mastocytoma cell is selected from the group consisting of MST cells, P815 cells, MC/9 cells, SI/SI4 cells, 10P2 cells, 11P0-1 cells, and 10P12 cells. In some cases, the cell is deficient for a heparan sulfate catabolic enzyme.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

An understanding of the features and advantages of the present invention will be obtained by reference to the following detailed description that sets forth illustrative embodiments, in which the principles of the invention are utilized, and the accompanying drawings of which:

FIG. 1A shows representative heparan sulfate and chondroitin sulfate structures with ATIII binding pentasaccharide and enzymes responsible for heparan sulfate modification and chondroitin sulfate polymerization shown.

FIG. 1B shows heparan sulfate purified from cultured cells. The heparan sulfate and unfractionated heparin were digested with heparin lyases and disaccharide composition was determined by LCMS. The abundance of sulfate groups in each position of the polysaccharide was determined from the disaccharide composition data.

FIG. 1C shows heparan sulfate and chondroitin sulfate purified from MST cells and their relative abundance as determined by LCMS.

FIG. 2A shows gene expression analysis of RNA isolated from cultured MST cells. cDNA was prepared using reverse transciptase and RNAseq analysis was performed to determine gene expression. The relative expression of each transcript was calculated as fragments per kilobase of transcript per million mapped reads. Genes responsible for CS biosynthesis are shown in this figure.

FIG. 2B shows five MST17 colonies with Csgalnact1, Csgalnact2 and Chsy1 mutated were cultured under identical conditions. Heparan sulfate was purified and quantified from the cell pellet of each culture.

FIG. S1A-S1F shows LCMS analysis of heparan sulfate (FIG. S1A, S1C, S1E) or chondroitin sulfate (FIG. S1B, S1D, S1F) disaccharides. Glycosaminoglycans were purified from MST cells (FIG. S1C, S1D) or MST17B10 cells (FIG. S1E, S1F) and were digested with heparin lyases (FIG. SIC, S1E) or chondroitinase ABC (FIG. S1D, S1F). The resulting disaccharides were analyzed by LCMS with total ion current gated to include only masses of the disaccharides.

FIG. 3A shows results from bone marrow derived cells (BMDC) differentiated into mast cells over three weeks. Heparan sulfate was purified from precursor and differentiated cells and sulfate content was determined by LCMS on the heparin lyase digested disaccharides.

FIG. 3B shows results of RNA that was collected from cultured bone marrow derived cells, differentiated mast cells and MST cells. cDNA was made and relative gene expression for heparan sulfate biosynthetic enzymes was determined by RNAseq.

FIG. S2A-S2B shows results from RNA that was collected from cultured bone marrow derived cells, differentiated mast cells and MST cells. cDNA was made and relative gene expression for enzymes and transporters responsible for nucleotide sugar metabolism (FIG. S2A) and proteoglycan core proteins (FIG. S2B) was determined by RNAseq.

FIG. 4A shows lineage of engineered cell lines depicted. MST was targeted by CRISPR to knock out Csgalnact1, Csgalnact2 and Chsy1 creating the MST17 population. Limiting dilution cloning yielded MST17B10, which was subsequently transduced to overexpress combinations of sulfotransferases. Transductions depicted are defined in the table.

FIG. 4B shows results from single cell colonies analyzed from transduction of MST17B10. Colonies were initially genotyped by PCR. The anti-Xa activity created by the colony in the well and normalized by protein content of the lysate (for a subset of the colonies) was determined from purified HS.

FIG. 5A shows HS was purified from the cell pellet and the conditioned medium of candidate cell lines grown in DMEM/F12+15% FBS or CDM4NS0 and quantified using the carbazole assay.

FIG. 5B shows disaccharide analysis was performed to determine the sulfate content.

FIG. 5C shows the anticoagulant potency was determined using the anti-Xa activity assay.

FIG. 5D shows the total anti-FXa activity yield from HS purified from both the cell pellet and the conditioned medium was determined using the carbazole assay and the anti-Xa activity assay. Samples were assayed independently two or three times each.

FIG. 6A shows B 3.E cells were grown in various medium. Integrated viable cell density was determined over 7 days.

FIG. 6B shows heparan sulfate was purified from these cells and total yield per volume of culture was determined by carbazole assay.

FIG. 6C shows MST38 cells were grown for 7 days in DMEM/F12+15% FBS or in serum free medium. Integrated viable cell density was determine over the duration of the culture.

FIG. 6D shows the anti-FXa activity of purified heparan sulfate was determined. The dotted line represents the USP anti-Xa requirement for unfractionated heparin. Each experiment was performed three times.

FIG. 7A shows heparan sulfate was purified from the cell pellet of B 7A cells grown in CDM4NS0. Sulfate content (or disaccharide composition) was determined by UPLC analysis.

FIG. 7B shows results of anti-Xa activity assay with PBS, MST38, and heparin.

FIG. 8A shows a standard curve for the Factor Xa activity assay (anticoagulant activity).

FIG. 8B shows that anticoagulant activity was reduced relative to the parent cell line (2.7A) but was still over 150 U/mg.

FIG. 8C shows the results of a PF4 competition binding assay.

FIG. 9A shows that cell line 2.7A-C20 (that overexpresses the Sulf-2 gene) maintains high antifactor Xa activity.

FIG. 9B shows the results of a PF4 competition binding assay.

### DETAILED DESCRIPTION

The anticoagulant potency of heparin was discovered more than 100 years ago. It has since been used for decades as a life-saving drug to control blood coagulation during renal dialysis, cardiac surgery and in treatment of deep vein thrombosis and disseminated intravascular coagulopathies. Its importance is clearly evident by its inclusion on the World Health Organization's list of “essential medicines” and by its widespread use, as hundreds of thousands of doses are administered daily world-wide. Heparin is a natural product derived primarily from porcine intestine with over 50% of the world's supply sourced as a byproduct of pork production.

Sourcing heparin from a single species puts the world supply at risk of disease epidemics in the pig population. In 2007, a highly infectious porcine virus (so-called blue ear disease) caused massive outbreaks of porcine reproductive and respiratory syndrome, drastically decreased the availability of pig products and resulted in a worldwide heparin shortage. Adulteration of heparin with non-naturally occurring over-sulfated chondroitin sulfate led to over 200 deaths worldwide and more than 800 reports of serious hypertension and adverse allergic reactions in 2008. The Food and Drug Administration in the United States continues to address concerns about foreign heparin production and its quality control. The vulnerability of the heparin supply chain continues to be a concern, especially in the wake of the current African Swine Fever epidemic that has claimed over 32% of China's pig herd since August 2018. The frequency of porcine epidemics suggests that the supply of animal sourced heparin will continue to be at risk.

As a solution to this problem, the FDA is considering the reintroduction of heparin from bovine tissues as an alternative source; however, there are currently no facilities in the US with the capacity to produce the necessary quantity and quality of material. Bovine heparin was discontinued in the US in the 1990s due to concerns over Bovine Spongiform Encephalopathy (BSE). BSE has all but been eliminated but bovine heparin is still at risk of contamination from bovine disease agents and has lower anticoagulant activity compared to porcine heparin.

An alternative solution to porcine and bovine heparin is to source the material in engineered cells. Ultimately, recombinant heparin generated in this way could replace animal derived heparin entirely if problems related to the quality and quantity of recombinant heparin can be solved. Unlike proteins expressed by a single gene, heparin, a polysaccharide, is produced in a metabolic pathway involving over 20 enzymes. Heparin is a form of heparan sulfate (HS), which is produced by all types of animal cells, but differs in the degree of sulfation and the level of anticoagulant activity. Heparin is made exclusively in granulated connective tissue type mast cells. Unfortunately, mast cells are difficult to propagate in cell culture and therefore are not appropriate for commercial production. In contrast, heparan sulfate can be produced in many types of cells used for recombinant protein production. Genetic manipulation of the cells in turn allows bioengineering of the structure of heparan sulfate, with the possibility of engineering cells to produce material that resembles pharmaceutical heparin.

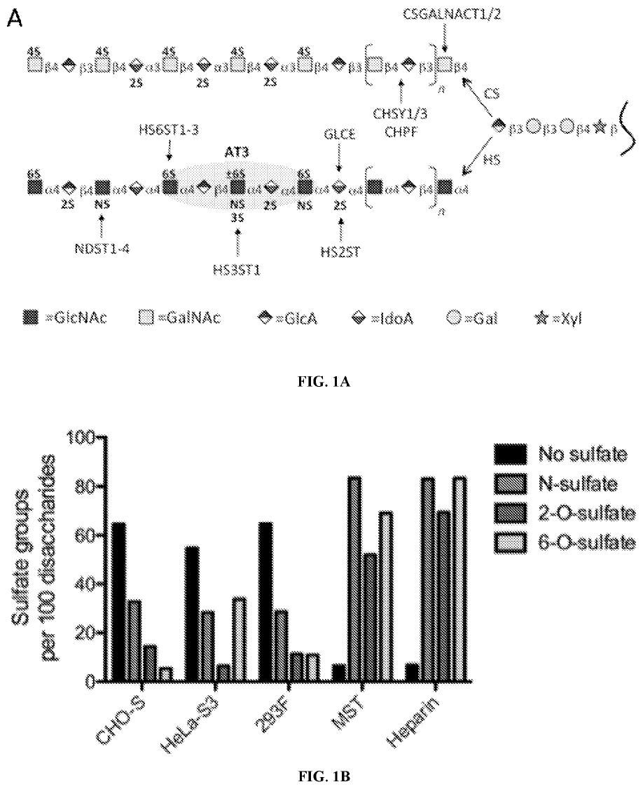

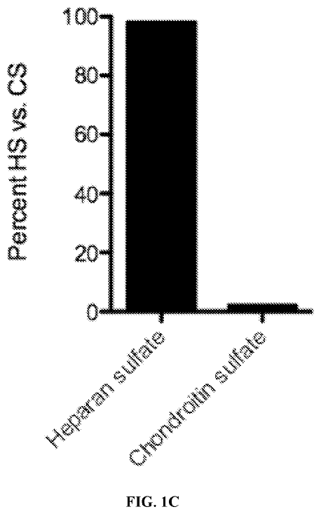

Heparin and HS are structurally similar consisting of a polysaccharide chain backbone composed of repeating alternating residues of α1,4-linked N-acetyl-D-glucosamine (GlcNAc) bound to β1,4-linked D-glucuronic acid residues. As the chains assemble, they undergo modifications that include N-deacetylation and N-sulfation of subsets of N-acetylglucosamine residues (catalyzed by 4 members of the NDST family), C5 epimerization of the D-glucuronic acid residues to L-iduronic acid (catalyzed by GLCE) (12), O-sulfation of uronic acids (catalyzed by HS2ST) and glucosamine residues by glucosaminyl-6-O-sulfotransferases (HS6ST, 3 isozymes) and glucosaminyl-3-O-sulfotransferase (HS3ST, 7 isozymes) (FIG. 1A). Modification of the polysaccharide chains does not go to completion. Heparin and HS are therefore heterogeneous mixtures of polydisperse and variably modified chains. Top down analysis of the sequence and domain structure of the chains is quite complex. Instead, bottom up analyses of the chains are possible by digestion of the chains into the disaccharides with bacterial heparin lyases or nitrous acid followed by chromatographic or electrophoretic separation of the differentially sulfated disaccharides. The rules that determine the individual sulfation reactions are partially understood e.g., N-sulfation precedes O-sulfation, iduronic acid is 2-O-sulfated much more frequently than glucuronic acid, 2-O-sulfate locks the uronic acid epimer in the gluco configuration, and sulfation levels appear to be determined by the expression levels of the corresponding sulfotransferases.

Heparin is a highly sulfated form of HS, in which many of the disaccharides contain iduronic acid and N- and O-sulfate groups. Anticoagulant activity depends of the presence of 3-O-sulfated N-sulfoglucosamine residues positioned within a preferred pentasaccharide sequence (GlcNAc6S-GlcA-GlcNS3S±6S-IdoA2S-GlcNS6S) (FIG. 1A). The presence of this pentasaccharide in heparin or in HS confers the capacity to bind to antithrombin, which results in a striking enhancement in the ability of antithrombin to inactivate Factors IIa (thrombin), Factor Xa and other activated proteases in the coagulation cascade. Thus, one strategy for engineering cells to increase the antithrombin binding pentasaccharide is to increase the expression levels of the relevant sulfotransferases. Previous attempts using this strategy have met with limited success (Baik, J. Y., et al. (2012) Metabolic engineering of Chinese hamster ovary cells: towards a bioengineered heparin. Metab Eng 14, 81-90; Gasimli, L., et al. (2014) Bioengineering murine mastocytoma cells to produce anticoagulant heparin. Glycobiology 24, 272-280; Datta, P., et al. (2013) Bioengineered Chinese hamster ovary cells with Golgi-targeted 3-O-sulfotransferase-1 biosynthesize heparan sulfate with an antithrombin-binding site. J Biol Chem 288, 37308-37318).

Provided herein are engineered cell lines that produce highly sulfated heparin/HS polysaccharide chains with anticoagulant activities and other properties comparable to pharmaceutical heparin. Specifically, a mastocytoma cell line is provided that naturally produces highly sulfated HS, but which lacked anticoagulant activity, is engineered to eliminate production of contaminating chondroitin sulfate (CS) and to produce highly anticoagulant HS with a composition approximating fractionated porcine heparin. In some cases, the engineered cell line is adapted to serum free medium.

### Methods of Producing Heparin and Heparan Sulfate

Provided herein are methods of producing heparin and heparan sulfate in cultured cells and isolating the heparin or heparan sulfate from the cells. Exemplary cultured cells for such methods include mastocytoma cell lines, such as MST cells, P815 cells, MC/9 cells SI/SI4 cells, 10P2 cells, 11P0-1 cells and 10P12 cells; RT4 cells; 682B cells; 751G cells; 1016T cells; KK-47; MGH-U1; MHG-U2; MGH-U3; MGH-U4; and basophil neoplastic cell lines. Specifically contemplated herein are genetically modified cell lines comprising gene deletions and/or gene overexpression to optimize the amount and type of heparin or heparan sulfate produced by the cell lines.

Genetic modifications suitable in mastocytoma cells, MST cells, and basophil neoplastic cell lines herein increase the amount of heparin and/or heparan sulfate, decrease the amount of contaminating substances, and alter the properties of the heparin and/or heparan sulfate produced by the cell lines. In some cases, genetically modified cell lines are deficient in one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), heparan sulfate 2-O-sulfotransferase (Hs2st), and heparan sulfate C5-epimerase (GLCE). In some cases, the genetically modified cell line is deficient for a heparan sulfate catabolic enzyme. Non-limiting exemplary heparan sulfate catabolic enzyme comprise one or more of heparanase (HPSE), beta-glucoronidase (GUSB), sulfamidase (SGSH), heparan alpha-glucosaminide N-acetyltransferase (HGNAT), uronate-2-O-sulfatases (IDS and GDS), alpha-L-iduronidase (IDUA), and heparan sulfate 6-O-endosulfatase (SULF1-2). In addition, the genetically modified cell lines are contemplated to overexpresses one or more of heparanase, a protease, a heparan sulfate copolymerase, a sulfotransferase, an endosulfatase, and a proteoglycan protein core. Non-limiting exemplary proteases comprise one or more of matrix metalloproteases (MMP), MMP2, MMP3, MMP7, MMP9, MT1-MMP, MT3-MMP, ADAM17, ADAMTS1 and ADAMTS4, as well as trypsin, and chymotrypsin. Exemplary proteoglycan protein core include but are not limited to serglycin, a syndecan, a glypican, CD44 isoforms (CD44E), perlecan, collagen XVIII, and agrin, and recombinant versions thereof. In some cases, serglycin comprises a human serglycin, a pig serglycin, a mouse serglycin. In addition, it is contemplated that the number of GAG attachment sites in the serglycin is modified. Exemplary syndecans include but are not limited to a syndecan selected from the group consisting of syndecan-1, syndecan-2, syndecan-3, syndecan-4, and syndecan ectodomains. Non-limiting examples of glypicans include glypican 1, glypican 2, glypican 3, glypican 4, glypican 5, and glypican 6 and ectodomains.

Additional genetic modifications of mastocytoma cells, MST cells, and basophil neoplastic cell lines are provided in Table 1 and Table 2 below.

TABLE 1
Genetic Modifications for Cellular Production
of Heparin and Heparan Sulfate

| Gene KO | Gene KI | Enzymes |
|---|---|---|
| Chsy1 | | CS deficient |
| Chsy1 | | CS deficient |
| Hs2st | | HS 2-O-sulfation deficient |
| Chsy1 | | CS deficient |
| Glce | | HS epimerization deficient |
| Chsy1 | | CS deficient |
| Ndst1 | | NDST1-sulfation deficient |
| Chsy1 | | CS deficient |
| Ndst2 | | NDST2-sulfation deficient |
| Chsy1 | | CS deficient |
| Ndst1 | | NDST1-sulfation deficient |
| Ndst2 | | NDST2-sulfation deficient |
| Chsy1 | | CS deficient |
| Ndst3 | | NDST3-sulfation deficient |
| Chsy1 | | CS deficient |
| Ndst4 | | NDST4-sulfation deficient |
| Chsy1 | | CS deficient |
| Sulf1 | | Sulfatase 1 deficient |
| Chsy1 | | CS deficient |
| Sulf2 | | Sulfatase 2 deficient |
| Chsy1 | | CS deficient |
| Sulf1/2 | | Sulfatase 1 deficient |
| | | Sulfatase 2 deficient |
| Chsy1 | Ndst3 | CS deficient |
| Ndst1 | | NDST1-sulfation deficient |
| Ndst2 | | NDST2-sulfation deficient |
| | | NDST3-sulfation Added |
| Chsy1 | Ndst4 | CS deficient |
| Ndst1 | | NDST1-sulfation deficient |
| Ndst2 | | NDST2-sulfation deficient |
| | | NDST4-sulfation Added |
| Chsy1 | Hs6st1 | CS deficient |
| | | HS6ST1-sulfation Added |
| Chsy1 | Hs6st2 | CS deficient |
| | | HS6ST2-sulfation Added |
| Chsy1 | Hs6st3 | CS deficient |
| | | HS6ST3-sulfation Added |
| Chsy1 | Hs6st1 | CS deficient |
| | Hs6st2 | HS6ST1-sulfation Added |
| | | HS6ST2-sulfation Added |
| Chsy1 | Hs3st1 | CS deficient |
| | | HS3ST1-sulfation Added |
| Chsy1 | Hs3st2 | CS deficient |
| | | HS3ST-2sulfation Added |
| Chsy1 | Hs3st3a | CS deficient |
| | | HS3ST3a-sulfation Added |
| Chsy1 | Hs3st3b | CS deficient |
| | | HS3ST3b-sulfation Added |
| Chsy1 | Hs3st4 | CS deficient |
| | | HS3ST4-sulfation Added |
| Chsy1 | Hs3st5 | CS deficient |
| | | HS3ST5-sulfation Added |
| Chsy1 | Hs3st6 | CS deficient |
| | | HS 3ST6-sulfation Added |
| Chsy1 | Hs6st1 | CS deficient |
| | Hs6st2 | HS6ST1-sulfation Added |
| | Hs3st1 | HS6ST2-sulfation Added |
| | | HS3ST1-sulfation Added |
| Chsy1 | Hs6st1 | CS deficient |
| | Hs6st2 | HS6ST1-sulfation Added |
| | Hs3st2 | HS6ST2-sulfation Added |
| | | HS3ST2-sulfation Added |
| Chsy1 | Hs6st1 | CS deficient |
| | Hs6st2 | HS6ST1-sulfation Added |
| | Hs3st3a or 3b | HS6ST2-sulfation Added |
| | | HS3ST3-sulfation Added |
| Chsy1 | Hs6st1 | CS deficient |
| | Hs6st2 | HS6ST1-sulfation Added |
| | Hs3st4 | HS6ST2-sulfation Added |
| | | HS3ST4-sulfation Added |
| Chsy1 | Hs6st1 | CS deficient |
| | Hs6st2 | HS6ST1-sulfation Added |
| | Hs3st5 | HS6ST2-sulfation Added |
| | | HS3ST5-sulfation Added |
| Chsy1 | Hs6st1 | CS deficient |
| | Hs6st2 | HS6ST1-sulfation Added |
| | Hs3st6 | HS6ST2-sulfation Added |
| | | HS3ST6-sulfation Added |
| Chsy1 | Ndst3 | CS deficient |
| Ndst1 | Hs6st1 | NDST1-sulfation deficient |
| Ndst2 | | NDST2-sulfation deficient |
| | | NDST3-sulfation Added |
| | | HS6ST1-sulfation Added |
| Chsy1 | Ndst3 | CS deficient |
| Ndst1 | Hs6st2 | NDST1-sulfation deficient |
| Ndst2 | | NDST2-sulfation deficient |
| | | NDST3-sulfation Added |
| | | HS6ST2-sulfation Added |
| Chsy1 | Ndst3 | CS deficient |
| Ndst1 | Hs6st3 | NDST1-sulfation deficient |
| Ndst2 | | NDST2-sulfation deficient |
| | | NDST3-sulfation Added |
| | | HS6ST3-sulfation Added |
| Chsy1 | Ndst4 | CS deficient |
| Ndst1 | Hs6st1 | NDST1-sulfation deficient |
| Ndst2 | | NDST2-sulfation deficient |
| | | NDST4-sulfation Added |
| | | HS6ST1-sulfation Added |
| Chsy1 | Ndst4 | CS deficient |
| Ndst1 | Hs6st2 | NDST1-sulfation deficient |
| Ndst2 | | NDST2-sulfation deficient |
| | | NDST4-sulfation Added |
| | | HS6ST2-sulfation Added |
| Chsy1 | Ndst4 | CS deficient |
| Ndst1 | Hs6st3 | NDST1-sulfation deficient |
| Ndst2 | | NDST2-sulfation deficient |
| | | NDST4-sulfation Added |
| | | HS6ST3-sulfation Added |
| Chsy1 | Ndst3 | CS deficient |
| Ndst1 | Hs3st1 | NDST1-sulfation deficient |
| Ndst2 | | NDST2-sulfation deficient |
| | | NDST3-sulfation Added |
| | | HS3ST1-sulfation Added |
| Chsy1 | Ndst3 | CS deficient |
| Ndst1 | Hs3st2 | NDST1-sulfation deficient |
| Ndst2 | | NDST2-sulfation deficient |
| | | NDST3-sulfation Added |
| | | HS3ST2-sulfation Added |
| Chsy1 | Ndst3 | CS deficient |
| Ndst1 | 3a or 3b | NDST1-sulfation deficient |
| Ndst2 | | NDST2-sulfation deficient |
| | | NDST3-sulfation Added |
| | | HS3ST3-sulfation Added |
| Chsy1 | Ndst3 | CS deficient |
| Ndst1 | Hs3st4 | NDST1-sulfation deficient |
| Ndst2 | | NDST2-sulfation deficient |
| | | NDST3-sulfation Added |
| | | HS3ST4-sulfation Added |
| Chsy1 | Ndst3 | CS deficient |
| Ndst1 | Hs3st5 | NDST1-sulfation deficient |
| Ndst2 | | NDST2-sulfation deficient |
| | | NDST3-sulfation Added |
| | | HS3ST5-sulfation Added |
| Chsy1 | Ndst3 | CS deficient |
| Ndst1 | Hs3st6 | NDST1-sulfation deficient |
| Ndst2 | | NDST2-sulfation deficient |
| | | NDST3-sulfation Added |
| | | HS3ST6-sulfation Added |
| Chsy1 | Ndst4 | CS deficient |
| Ndst1 | Hs3st1 | NDST1-sulfation deficient |
| Ndst2 | | NDST2-sulfation deficient |
| | | NDST4-sulfation Added |
| | | HS3ST1-sulfation Added |
| Chsy1 | Ndst4 | CS deficient |
| Ndst1 | Hs3st2 | NDST1-sulfation deficient |
| Ndst2 | | NDST2-sulfation deficient |
| | | NDST4-sulfation Added |
| | | HS3ST2-sulfation Added |
| Chsy1 | Ndst4 | CS deficient |
| Ndst1 | Hs3st3a or 3b | NDST1-sulfation deficient |
| Ndst2 | | NDST2-sulfation deficient |
| | | NDST4-sulfation Added |
| | | HS3ST3-sulfation Added |
| Chsy1 | Ndst4 | CS deficient |
| Ndst1 | Hs3st4 | NDST1-sulfation deficient |
| Ndst2 | | NDST2-sulfation deficient |
| | | NDST4-sulfation Added |
| | | HS3ST4-sulfation Added |
| Chsy1 | Ndst4 | CS deficient |
| Ndst1 | Hs3st5 | NDST1-sulfation deficient |
| Ndst2 | | NDST2-sulfation deficient |
| | | NDST4-sulfation Added |
| | | HS3ST5-sulfation Added |
| Chsy1 | Ndst4 | CS deficient |
| Ndst1 | Hs3st6 | NDST1-sulfation deficient |
| Ndst2 | | NDST2-sulfation deficient |
| | | NDST4-sulfation Added |
| | | HS3ST6-sulfation Added |
| Chsy1 | Ndst3 | CS deficient |
| Ndst1 | Hs6st1/2 | NDST1-sulfation deficient |
| Ndst2 | Hs3st1 | NDST2-sulfation deficient |
| | | NDST3-sulfation Added |
| | | HS6ST1/2-sulfation Added |
| | | HS3ST1-sulfation Added |
| Chsy1 | Ndst3 | CS deficient |
| Ndst1 | Hs6st1/2 | NDST1-sulfation deficient |
| Ndst2 | Hs3st2 | NDST2-sulfation deficient |
| | | NDST3-sulfation Added |
| | | HS6ST1/2-sulfation Added |
| | | HS3ST2-sulfation Added |
| Chsy1 | Ndst3 | CS deficient |
| Ndst1 | Hs6st1/2 | NDST1-sulfation deficient |
| Ndst2 | Hs3st3a or 3b | NDST2-sulfation deficient |
| | | NDST3-sulfation Added |
| | | HS6ST1/2-sulfation Added |
| | | HS3ST3-sulfation Added |
| Chsy1 | Ndst3 | CS deficient |
| Ndst1 | Hs6st1/2 | NDST1-sulfation deficient |
| Ndst2 | Hs3stT4 | NDST2-sulfation deficient |
| | | NDST3-sulfation Added |
| | | HS6ST1/2-sulfation Added |
| | | HS3ST4-sulfation Added |
| Chsy1 | Ndst3 | CS deficient |
| Ndst1 | Hs6st1/2 | NDST1-sulfation deficient |
| Ndst2 | Hs3st5 | NDST2-sulfation deficient |
| | | NDST3-sulfation Added |
| | | HS6ST1/2-sulfation Added |
| | | HS3ST5-sulfation Added |
| Chsy1 | Ndst3 | CS deficient |
| Ndst1 | Hs6st1/2 | NDST1-sulfation deficient |
| Ndst2 | Hs3st6 | NDST2-sulfation deficient |
| | | NDST3-sulfation Added |
| | | HS6ST1/2-sulfation Added |
| | | HS3ST6-sulfation Added |
| Chsy1 | Ndst4 | CS deficient |
| Ndst1 | Hs6stT1/2 | NDST1-sulfation deficient |
| Ndst2 | Hs3st1 | NDST2-sulfation deficient |
| | | NDST4-sulfation Added |
| | | HS6ST1/2-sulfation Added |
| | | HS3ST1-sulfation Added |
| Chsy1 | Ndst3 | CS deficient |
| Ndst1 | Hs6st1/2 | NDST1-sulfation deficient |
| Ndst2 | Hs3st2 | NDST2-sulfation deficient |
| | | NDST4-sulfation Added |
| | | HS6ST1/2-sulfation Added |
| | | HS3ST2-sulfation Added |
| Chsy1 | Ndst3 | CS deficient |
| Ndst1 | Hs6st1/2 | NDST1-sulfation deficient |
| Ndst2 | Hs3st3a or 3b | NDST2-sulfation deficient |
| | | NDST4-sulfation Added |
| | | HS6ST1/2-sulfation Added |
| | | HS3ST3-sulfation Added |
| Chsy1 | Ndst3 | CS deficient |
| Ndst1 | Hs6st1/2 | NDST1-sulfation deficient |
| Ndst2 | HSs3st4 | NDST2-sulfation deficient |
| | | NDST4-sulfation Added |
| | | HS6ST1/2-sulfation Added |
| | | HS3ST4-sulfation Added |
| Chsy1 | Ndst3 | CS deficient |
| Ndst1 | Hs6st1/2 | NDST1-sulfation deficient |
| Ndst2 | Hs3st5 | NDST2-sulfation deficient |
| | | NDST4-sulfation Added |
| | | HS6ST1/2-sulfation Added |
| | | HS3ST5-sulfation Added |
| Chsy1 | Ndst3 | CS deficient |
| Ndst1 | Hs6st1/2 | NDST1-sulfation deficient |
| Ndst2 | Hs3st6 | NDST2-sulfation deficient |
| | | NDST4-sulfation Added |
| | | HS6ST1/2-sulfation Added |
| | | HS3ST6-sulfation Added |
| Chsy1 | Ndst1 | CS deficient |
| | | NDST1 over-sulfation |
| Chsy1 | Ndst2 | CS deficient |
| | | NDST2 over-sulfation |
| Chsy1 | Ndst1 & 2 | CS deficient |
| | | NDST1 & 2 over-sulfation |
| Chsy1 | Ndst1 | CS deficient |
| | Hs2st | NDST over-sulfation |
| | | HS2ST over-sulfation |
| Chsy1 | Ndst2 | CS deficient |
| | Hs2st | NDST2 over-sulfation |
| | | HS2ST over-sulfation |
| Chsy1 | Ndst1 & 2 | CS deficient |
| | Hs2st | NDST1 & 2 over-sulfation |
| | | HS2ST over-sulfation |
| Chsy1 | Ndst1 | CS deficient |
| | Glce | NDST1 over-sulfation |
| | | Epimerase over-epimerization |
| Chsy1 | Ndst2 | CS deficient |
| | Glce | NDST2 over-sulfation |
| | | Epimerase over-epimerization |
| Chsy1 | Ndst1 & 2 | CS deficient |
| | Glce | NDST1 & 2 over-sulfation |
| | | Epimerase over-epimerization |
| Chsy1 | Ndst1 | CS deficient |
| | Hs2st | NDST1 over-sulfation |
| | Glce | HS2ST over-sulfation |
| | | Epimerase over-epimerization |
| Chsy1 | Ndst2 | CS deficient |
| | Hs2st | NDST2 over-sulfation |
| | Glce | HS2ST over-sulfation |
| | | Epimerase over-epimerization |
| Chsy1 | Ndst1 & 2 | CS deficient |
| | Hs2st | NDST1 & 2 over-sulfation |
| | Glce | HS2ST over-sulfation |
| | | Epimerase over-epimerization |
| Chsy1 | Ndst1 | CS deficient |
| | Hs6st 1, 2 or 1 & 2 | NDST1 over-sulfation |
| | | HS6ST over-sulfation |
| Chsy1 | Ndst2 | CS deficient |
| | Hs6st 1, 2 or 1 & 2 | NDST2 over-sulfation |
| Chsy1 | Ndst1 & 2 | CS deficient |
| | Hs6st 1, 2 or 1 & 2 | NDST1 & 2 over-sulfation |
| Chsy1 | Ndst1 | CS deficient |
| | HS2st | NDST over-sulfation |
| | Hs6st 1, 2 or 1 & 2 | HS2ST over-sulfation |
| | | HS6ST over-sulfation |
| Chsy1 | Ndst2 | CS deficient |
| | Hs2st | NDST2 over-sulfation |
| | Hs6st 1, 2 or 1 & 2 | HS2ST over-sulfation |
| Chsy1 | Ndst1 & 2 | CS deficient |
| | Hs2st | NDST1 & 2 over-sulfation |
| | Hs6st 1, 2 or 1 & 2 | HS2ST over-sulfation |
| Chsy1 | Ndst1 | CS deficient |
| | Glce | NDST1 over-sulfation |
| | Hs6st 1, 2 or 1 & 2 | Epimerase over-epimerization |
| Chsy1 | Ndst2 | CS deficient |
| | Glce | NDST2 over-sulfation |
| | Hs6st 1, 2 or 1 & 2 | Epimerase over-epimerization |
| Chsy1 | Ndst1 & 2 | CS deficient |
| | Glce | NDST1 & 2 over-sulfation |
| | Hs6st 1, 2 or 1 & 2 | Epimerase over-epimerization |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | | HS6ST over-sulfation |
| Chsy1 | Ndst1 | CS deficient |
| | Hs3st | NDST1 over-sulfation |
| | | HS3ST over-sulfation |
| Chsy1 | Ndst2 | CS deficient |
| | Hs3st | NDST2 over-sulfation |
| | | HS3ST over-sulfation |
| Chsy1 | Ndst1 & 2 | CS deficient |
| | Hs3st | NDST1 & 2 over-sulfation |
| | | HS3ST over-sulfation |
| Chsy1 | Ndst1 | CS deficient |
| | Hs2st | NDST over-sulfation |
| | Hs3st | HS2ST over-sulfation |
| | | HS3ST over-sulfation |
| Chsy1 | Ndst2 | CS deficient |
| | Hs2st | NDST2 over-sulfation |
| | Hs3st | HS2ST over-sulfation |
| | | HS3ST over-sulfation |
| Chsy1 | Ndst1 & 2 | CS deficient |
| | Hs2st | NDST1 & 2 over-sulfation |
| | Hs3st | HS2ST over-sulfation |
| | | HS3ST over-sulfation |
| Chsy1 | Ndst1 | CS deficient |
| | Glce | NDST1 over-sulfation |
| | Hs3st | Epimerase over-epimerization |
| | | HS3ST over-sulfation |
| Chsy1 | Ndst2 | CS deficient |
| | Glce | NDST2 over-sulfation |
| | Hs3st | Epimerase over-epimerization |
| | | HS3ST over-sulfation |
| Chsy1 | Ndst1 & 2 | CS deficient |
| | Glce | NDST1 & 2 over-sulfation |
| | Hs3st | Epimerase over-epimerization |
| | | HS3ST over-sulfation |
| Chsy1 | Ndst1 | CS deficient |
| | Hs2st | NDST over-sulfation |
| | Glce | HS2ST over-sulfation |
| | Hs3st | Epimerase over-epimerization |
| | | HS3ST over-sulfation |
| Chsy1 | Ndst2 | CS deficient |
| | Hs2st | NDST2 over-sulfation |
| | Glce | HS2ST over-sulfation |
| | Hs3st | Epimerase over-epimerization |
| | | HS3ST over-sulfation |
| Chsy1 | Ndst1 & 2 | CS deficient |
| | Hs2st | NDST1 & 2 over-sulfation |
| | Glce | HS2ST over-sulfation |
| | Hs3st | Epimerase over-epimerization |
| | | HS3ST over-sulfation |
| Chsy1 | Ndst1 | CS deficient |
| | Hs6st 1, 2 or 1 & 2 | NDST1 over-sulfation |
| | Hs3st | HS6ST over-sulfation |
| | | HS3ST over-sulfation |
| Chsy1 | Ndst2 | CS deficient |
| | Hs6st 1, 2 or 1 & 2 | NDST2 over-sulfation |
| | Hs3st | HS3ST over-sulfation |
| | | HS3ST over-sulfation |
| Chsy1 | Ndst1 & 2 | CS deficient |
| | Hs6st 1, 2 or 1 & 2 | NDST1 & 2 over-sulfation |
| | Hs3st | HS3ST over-sulfation |
| Chsy1 | Ndst1 | CS deficient |
| | HS2st | NDST over-sulfation |
| | Hs6st 1, 2 or 1 & 2 | HS2ST over-sulfation |
| | Hs3st | HS6ST over-sulfation |
| | | HS3ST over-sulfation |
| Chsy1 | Ndst2 | CS deficient |
| | Hs2st | NDST2 over-sulfation |
| | Hs6st 1, 2 or 1 & 2 | HS2ST over-sulfation |
| | Hs3st | HS3ST over-sulfation |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Hs3st | HS3ST over-sulfation |
| Chsy1 | Ndst1 | CS deficient |
| | Glce | NDST1 over-sulfation |
| | Hs6st 1, 2 or 1 & 2 | Epimerase over-epimerization |
| | Hs3st | HS3ST over-sulfation |
| Chsy1 | Ndst2 | CS deficient |
| | Glce | NDST2 over-sulfation |
| | Hs6st 1, 2 or 1 & 2 | Epimerase over-epimerization |
| | Hs3st | HS3ST over-sulfation |
| Chsy1 | Ndst1 & 2 | CS deficient |
| | Glce | NDST1 & 2 over-sulfation |
| | Hs6st 1, 2 or 1 & 2 | Epimerase over-epimerization |
| | Hs3st | HS3ST over-sulfation |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Hs3st | HS3ST over-sulfation |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Hs3st | HS3ST over-sulfation |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Hs3st | HS6ST over-sulfation |
| | | HS3ST over-sulfation |
| Chsy1 | Ndst1 | CS deficient |
| | Srgn | NDST1 over-sulfation |
| | | Increased expression of serglycin |
| Chsy1 | Ndst2 | CS deficient |
| | Srgn | NDST2 over-sulfation |
| | | Increased expression of serglycin |
| Chsy1 | Ndst1 & 2 | CS deficient |
| | Srgn | NDST1 & 2 over-sulfation |
| | | Increased expression of serglycin |
| Chsy1 | Ndst1 | CS deficient |
| | Hs2st | NDST over-sulfation |
| | Srgn | HS2ST over-sulfation |
| | | Increased expression of serglycin |
| Chsy1 | Ndst2 | CS deficient |
| | Hs2st | NDST2 over-sulfation |
| | Srgn | HS2ST over-sulfation |
| | | Increased expression of serglycin |
| Chsy1 | Ndst1 & 2 | CS deficient |
| | Hs2st | NDST1 & 2 over-sulfation |
| | Srgn | HS2ST over-sulfation |
| | | Increased expression of serglycin |
| Chsy1 | Ndst1 | CS deficient |
| | Glce | NDST1 over-sulfation |
| | Srgn | Epimerase over-epimerization |
| | | Increased expression of serglycin |
| Chsy1 | Ndst2 | CS deficient |
| | Glce | NDST2 over-sulfation |
| | Srgn | Epimerase over-epimerization |
| | | Increased expression of serglycin |
| Chsy1 | Ndst1 & 2 | CS deficient |
| | Glce | NDST1 & 2 over-sulfation |
| | Srgn | Epimerase over-epimerization |
| | | Increased expression of serglycin |
| Chsy1 | Ndst1 | CS deficient |
| | Hs2st | NDST over-sulfation |
| | Glce | HS2ST over-sulfation |
| | Srgn | Epimerase over-epimerization |
| | | Increased expression of serglycin |
| Chsy1 | Ndst2 | CS deficient |
| | Hs2st | NDST2 over-sulfation |
| | Glce | HS2ST over-sulfation |
| | Srgn | Epimerase over-epimerization |
| | | Increased expression of serglycin |
| Chsy1 | Ndst1 & 2 | CS deficient |
| | Hs2st | NDST1 & 2 over-sulfation |
| | Glce | HS2ST over-sulfation |
| | Srgn | Epimerase over-epimerization |
| | | Increased expression of serglycin |
| Chsy1 | Ndst1 | CS deficient |
| | Hs6st 1, 2 or 1 & 2 | NDST1 over-sulfation |
| | Srgn | HS6ST over-sulfation |
| | | Increased expression of serglycin |
| Chsy1 | Ndst2 | CS deficient |
| | Hs6st 1, 2 or 1 & 2 | NDST2 over-sulfation |
| | Srgn | HS6ST over-sulfation |
| | | Increased expression of serglycin |
| Chsy1 | Ndst1 & 2 | CS deficient |
| | Hs6st 1, 2 or 1 & 2 | NDST1 & 2 over-sulfation |
| | Srgn | HS6ST over-sulfation |
| | | Increased expression of serglycin |
| Chsy1 | Ndst1 | CS deficient |
| | HS2st | NDST over-sulfation |
| | Hs6st 1, 2 or 1 & 2 | HS2ST over-sulfation |
| | Srgn | HS6ST over-sulfation |
| | | Increased expression of serglycin |
| Chsy1 | Ndst2 | CS deficient |
| | Hs2st | NDST2 over-sulfation |
| | Hs6st 1, 2 or 1 & 2 | HS2ST over-sulfation |
| | Srgn | Increased expression of serglycin |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Srgn | Increased expression of serglycin |
| Chsy1 | Ndst1 | CS deficient |
| | Glce | NDST1 over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst2 | CS deficient |
| | Glce | NDST2 over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst1 & 2 | CS deficient |
| | Glce | NDST1 & 2 over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Srgn | HS6ST over-sulfation |
| | | Increased expression of serglycin |
| Chsy1 | Ndst1 | CS deficient |
| | Hs3st | NDST1 over-sulfation |
| | Srgn | HS3ST over-sulfation |
| | | Increased expression of serglycin |
| Chsy1 | Ndst2 | CS deficient |
| | Hs3st | NDST2 over-sulfation |
| | Srgn | HS3ST over-sulfation |
| | | Increased expression of serglycin |
| Chsy1 | Ndst1 & 2 | CS deficient |
| | Hs3st | NDST1 & 2 over-sulfation |
| | Srgn | HS3ST over-sulfation |
| | | Increased expression of serglycin |
| Chsy1 | Ndst1 | CS deficient |
| | Hs2st | NDST over-sulfation |
| | Hs3st | HS2ST over-sulfation |
| | Srgn | HS3ST over-sulfation |
| | | Increased expression of serglycin |
| Chsy1 | Ndst2 | CS deficient |
| | Hs2st | NDST2 over-sulfation |
| | Hs3st | HS2ST over-sulfation |
| | Srgn | HS3ST over-sulfation |
| | | Increased expression of serglycin |
| Chsy1 | Ndst1 & 2 | CS deficient |
| | Hs2st | NDST1 & 2 over-sulfation |
| | Hs3st | HS2ST over-sulfation |
| | Srgn | HS3ST over-sulfation |
| | | Increased expression of serglycin |
| Chsy1 | Ndst1 | CS deficient |
| | Glce | NDST1 over-sulfation |
| | Hs3st | Epimerase over-epimerization |
| | Srgn | HS3ST over-sulfation |
| | | Increased expression of serglycin |
| Chsy1 | Ndst2 | CS deficient |
| | Glce | NDST2 over-sulfation |
| | Hs3st | Epimerase over-epimerization |
| | Srgn | HS3ST over-sulfation |
| | | Increased expression of serglycin |
| Chsy1 | Ndst1 & 2 | CS deficient |
| | Glce | NDST1 & 2 over-sulfation |
| | Hs3st | Epimerase over-epimerization |
| | Srgn | HS3ST over-sulfation |
| | | Increased expression of serglycin |
| Chsy1 | Ndst1 | CS deficient |
| | Hs2st | NDST over-sulfation |
| | Glce | HS2ST over-sulfation |
| | Hs3st | Epimerase over-epimerization |
| | Srgn | HS3ST over-sulfation |
| | | Increased expression of serglycin |
| Chsy1 | Ndst2 | CS deficient |
| | Hs2st | NDST2 over-sulfation |
| | Glce | HS2ST over-sulfation |
| | Hs3st | Epimerase over-epimerization |
| | Srgn | HS3ST over-sulfation |
| | | Increased expression of serglycin |
| Chsy1 | Ndst1 & 2 | CS deficient |
| | Hs2st | NDST1 & 2 over-sulfation |
| | Glce | HS2ST over-sulfation |
| | Hs3st | Epimerase over-epimerization |
| | Srgn | HS3ST over-sulfation |
| | | Increased expression of serglycin |
| Chsy1 | Ndst1 | CS deficient |
| | Hs6st 1, 2 or 1 & 2 | NDST1 over-sulfation |
| | Hs3st | HS6ST over-sulfation |
| | Srgn | HS3ST over-sulfation |
| | | Increased expression of serglycin |
| Chsy1 | Ndst2 | CS deficient |
| | Hs6st 1, 2 or 1 & 2 | NDST2 over-sulfation |
| | Hs3st | HS3ST over-sulfation |
| | Srgn | HS3ST over-sulfation |
| | | Increased expression of serglycin |
| Chsy1 | Ndst1 & 2 | CS deficient |
| | Hs6st 1, 2 or 1 & 2 | NDST1 & 2 over-sulfation |
| | Hs3st | HS6ST over-sulfation |
| | Srgn | HS3ST over-sulfation |
| | | Increased expression of serglycin |
| Chsy1 | Ndst1 | CS deficient |
| | HS2st | NDST over-sulfation |
| | Hs6st 1, 2 or 1 & 2 | HS2ST over-sulfation |
| | Hs3st | HS6ST over-sulfation |
| | Srgn | HS3ST over-sulfation |
| | | Increased expression of serglycin |
| Chsy1 | Ndst2 | CS deficient |
| | Hs2st | NDST2 over-sulfation |
| | Hs6st 1, 2 or 1 & 2 | HS6ST over-sulfation |
| | Hs3st | HS2ST over-sulfation |
| | Srgn | HS3ST over-sulfation |
| | | Increased expression of serglycin |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Hs3st | HS6ST over-expression |
| | Srgn | HS3ST over-sulfation |
| | | Increased expression of serglycin |
| Chsy1 | Ndst1 | CS deficient |
| | Glce | NDST1 over-sulfation |
| | Hs6st 1, 2 or 1 & 2 | Epimerase over-epimerization |
| | Hs3st | HS6ST over-expression |
| | Srgn | HS3ST over-sulfation |
| | | Increased expression of serglycin |
| Chsy1 | Ndst2 | CS deficient |
| | Glce | NDST2 over-sulfation |
| | Hs6st 1, 2 or 1 & 2 | Epimerase over-epimerization |
| | Hs3st | HS6ST over-expression |
| | Srgn | HS3ST over-sulfation |
| | | Increased expression of serglycin |
| Chsy1 | Ndst1 & 2 | CS deficient |
| | Glce | NDST1 & 2 over-sulfation |
| | Hs6st 1, 2 or 1 & 2 | Epimerase over-epimerization |
| | Hs3st | HS6ST oversulfation |
| | Srgn | HS3ST over-sulfation |
| | | Increased expression of serglycin |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | | Increased expression of serglycin |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Hs3st | HS6ST over-expression |
| | Srgn | HS3ST over-sulfation |
| | | Increased expression of serglycin |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | | Increased expression of serglycin |
| Chsy1 | Ndst1 | CS deficient |
| | Srgn | NDST1 over-sulfation |
| | EXT2, 1, L3 | Increased expression of serglycin |
| | | HS over-polymerization |
| Chsy1 | Ndst2 | CS deficient |
| | Srgn | NDST2 over-sulfation |
| | EXT2, 1, L3 | Increased expression of serglycin |
| | | HS over-polymerization |
| Chsy1 | Ndst1 & 2 | CS deficient |
| | Srgn | NDST1 & 2 over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Srgn | HS2ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Srgn | HS2ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Srgn | HS2ST over-sulfation |
| | EXT2, 1, L3 | Increased expression of serglycin |
| | | HS over-polymerization |
| Chsy1 | Ndst1 | CS deficient |
| | Glce | NDST1 over-sulfation |
| | Srgn | Epimerase over-epimerization |
| | EXT2, 1, L3 | Increased expression of serglycin |
| | | HS over-polymerization |
| Chsy1 | Ndst2 | CS deficient |
| | Glce | NDST2 over-sulfation |
| | Srgn | Epimerase over-epimerization |
| | EXT2, 1, L3 | Increased expression of serglycin |
| | | HS over-polymerization |
| Chsy1 | Ndst1 & 2 | CS deficient |
| | Glce | NDST1 & 2 over-sulfation |
| | Srgn | Epimerase over-epimerization |
| | EXT2, 1, L3 | Increased expression of serglycin |
| | | HS over-polymerization |
| Chsy1 | Ndst1 | CS deficient |
| | Hs2st | NDST over-sulfation |
| | Glce | HS2ST over-sulfation |
| | Srgn | Epimerase over-epimerization |
| | EXT2, 1, L3 | Increased expression of serglycin |
| | | HS over-polymerization |
| Chsy1 | Ndst2 | CS deficient |
| | Hs2st | NDST2 over-sulfation |
| | Glce | HS2ST over-sulfation |
| | Srgn | Epimerase over-epimerization |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Glce | HS2ST over-sulfation |
| | Srgn | Epimerase over-epimerization |
| | EXT2, 1, L3 | Increased expression of serglycin |
| | | HS over-polymerization |
| Chsy1 | Ndst1 | CS deficient |
| | Hs6st 1, 2 or 1 & 2 | NDST1 over-sulfation |
| | Srgn | HS6ST over-sulfation |
| | EXT2, 1, L3 | Increased expression of serglycin |
| | | HS over-polymerization |
| Chsy1 | Ndst2 | CS deficient |
| | Hs6st 1, 2 or 1 & 2 | NDST2 over-sulfation |
| | Srgn | HS6ST over-sulfation |
| | EXT2, 1, L3 | Increased expression of serglycin |
| | | HS over-polymerization |
| Chsy1 | Ndst1 & 2 | CS deficient |
| | Hs6st 1, 2 or 1 & 2 | NDST1 & 2 over-sulfation |
| | Srgn | HS6ST over-sulfation |
| | EXT2, 1, L3 | Increased expression of serglycin |
| | | HS over-polymerization |
| Chsy1 | Ndst1 | CS deficient |
| | Hs2st | NDST1 over-sulfation |
| | Hs6st 1, 2 or 1 & 2 | HS2ST over-sulfation |
| | Srgn | HS6ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Hs6st 1, 2 or 1 & 2 | HS2ST over-sulfation |
| | Srgn | HS6ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Srgn | HS6ST over-sulfation |
| | EXT2, 1, L3 | Increased expression of serglycin |
| | | HS over-polymerization |
| Chsy1 | Ndst1 | CS deficient |
| | Glce | NDST1 over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst2 | CS deficient |
| | Glce | NDST2 over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst1 & 2 | CS deficient |
| | Glce | NDST1 & 2 over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Srgn | HS6ST over-sulfation |
| | EXT2, 1, L3 | Increased expression of serglycin |
| | | HS over-polymerization |
| Chsy1 | Ndst1 | CS deficient |
| | Hs3st | NDST1 over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs3st | NDST2 over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs3st | NDST1 & 2 over-sulfation |
| | Srgn | HS3ST over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Hs3st | HS2ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Hs3st | HS2ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Hs3st | HS2ST over-sulfation |
| | Srgn | HS3ST over-sulfation |
| | EXT2, 1, L3 | Increased expression of serglycin |
| | | HS over-polymerization |
| Chsy1 | Ndst1 | CS deficient |
| | Glce | NDST1 over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst2 | CS deficient |
| | Glce | NDST2 over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Glce | NDST1 & 2 over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Glce | HS2ST over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Glce | HS2ST over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Glce | HS2ST over-sulfation |
| | Hs3st | Epimerase over-epimerization |
| | Srgn | HS3ST over-sulfation |
| | EXT2, 1, L3 | Increased expression of serglycin |
| | | HS over-polymerization |
| Chsy1 | Ndst1 | CS deficient |
| | Hs6st 1, 2 or 1 & 2 | NDST1 over-sulfation |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs6st 1, 2 or 1 & 2 | NDST2 over-sulfation |
| | Hs3st | HS3ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs6st 1, 2 or 1 & 2 | NDST1 & 2 over-sulfation |
| | Hs3st | HS3ST over-sulfation |
| | Srgn | Increased expression of serglycin |
| | EXT2, 1, L3 | HS over-polymerization |
| Chsy1 | Ndst1 | CS deficient |
| | HS2st | NDST over-sulfation |
| | Hs6st 1, 2 or 1 & 2 | HS2ST over-sulfation |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Hs6st 1, 2 or 1 & 2 | HS2ST over-sulfation |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Hs6st 1, 2 or 1 & 2 | HS2ST over-sulfation |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 | CS deficient |
| | Glce | NDST1 over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Glce | NDST2 over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Glce | NDST1 & 2 over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 | CS deficient |
| | Hs2st | NDST over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst2 | CS deficient |
| | Hs2st | NDST2 over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |
| Chsy1 | Ndst1 & 2 | CS deficient |
| | Hs2st | NDST1 & 2 over-sulfation |
| | Glce | HS2ST over-sulfation |
| | Hs6st 1, 2 or 1 & 2 | Epimerase over-epimerization |
| | Hs3st | HS6ST over-sulfation |
| | Srgn | HS3ST over-sulfation |
| | EXT2, 1, L3 | Increased expression of serglycin |
| | | HS over-polymerization |

TABLE 2
Genes for Genetically Modified Cells Lines Producing Heparin and Heparan Sulfate

| Gene (Accession | | |
|---|---|---|
| Number) | Protein Name | Enzymatic Activity |
| SLC26A2 | DTDST sulfate anion | Transport of sulfate across plasma |
| (NP_000103) | transporter 1 | membrane |
| PAPSS1 (Y10387) | PAPS synthetase-1 (ATP | PAPS formation |
| | sulfurylase/APS kinase 1) | KI: increase sulfation |
| | | KO: decrease sulfation |
| PAPSS2 | PAPS synthetase-2 (ATP | PAPS formation |
| (AF074331) | sulfurylase/APS kinase 2) | KI: increase sulfation |
| | | KO: decrease sulfation |
| AB107958 | PAPS transporter | Import of PAPS into Golgi |
| | | KI: increase sulfation |
| | | KO: decrease sulfation |
| SLC35B2 | PAPS transporter 1 | Import of PAPS into Golgi |
| (Q8TB61.1) | | KI: increase sulfation |
| | | KO: decrease sulfation |
| SLC35B3 | PAPS transporter 2 | Import of PAPS into Golgi |
| (Q9H1N7.1) | | KI: increase sulfation |
| | | KO: decrease sulfation |
| SLC35D1 | UDP-GlcA transporter | Import of UDP-GlcA/UDP-GalNAc |
| (NP_055954) | | KI: increase GAG production |
| | | KO: decrease GAG production |
| SLC35A3 | UDP-GlcNAc transporter | Import of UDP-GlcNAc |
| (NP_036375) | | KI: increase GAG production |
| | | KO: decrease GAG production |
| SLC35A2 | UDP-Gal transporter | Import of UDP-Gal |
| (P78381) | | KI: increase GAG production |
| | | KO: decrease GAG production |
| UGDH | UDP glucose dehydrogenase | Conversion of UDP-Glc to UDP-GlcA |
| (AAC36095) | | KI: increase GAG production |
| | | KO: decrease GAG production |
| UXS | UDP-glucose decarboxylase | Conversion of UDP-GlcA to UDP-Xyl |
| (AAK85410) | | KI: increase GAG production |
| | | KO: decrease GAG production |
| XTI | Xylosyltransferase I | Attachment of Xylose to Ser of core protein |
| (AJ277441) | | KI: increase GAG production |
| | | KO: decrease GAG production |
| XTII | Xylosyltransferase II | Attachment of Xylose to Ser of core protein |
| (AJ277442) | | KI: increase GAG production |
| | | KO: decrease GAG production |
| XGALT-1 | Galactosyltransferase I | Addition of Galβ4 to Xylβ4Ser |
| (AB028600) | (β4GalT7) | KI: increase GAG production |
| | | KO: decrease GAG production |
| GALT-2 | Galactosyltransferase II | Addition of Galβ3 to Galβ4Xyl |
| (AY050570) | (β3GalT6) | KI: increase GAG production |
| | | KO: decrease GAG production |
| GLCAT-1 | Glucuronosyltransferase I | Addition of GlcAβ3 to Galβ3Galβ4Xyl |
| (AB009598) | | KI: increase GAG production |
| | | KO: decrease GAG production |
| EXTL1 | α-N- | Addition of GlcNAcα4 to Galβ3Galβ4Xyl |
| (AH007206) | acetylglucosaminyltransferase I | KI: increase GAG production |
| | | KO: decrease GAG production |
| EXTL2 | α-N- | Addition of GlcNAcα4 to Galβ3Galβ4Xyl |
| (AF000416) | acetylglucosaminyltransferase I | KI: increase GAG production |
| | | KO: decrease GAG production |
| EXTL3 | α-N- | Addition of GlcNAcα4 to Galβ3Galβ4Xyl |
| (AF001690) | acetylglucosaminyltransferase I | KI: increase GAG production |
| | | KO: decrease GAG production |
| EXT1 | Exostosin glycosyltransferase 1 | GlcNAcα4 & GlcAβ4 |
| (S79639) | | transferases: HS copolymerase |
| | | KI: increase GAG production |
| | | KO: decrease GAG production |
| EXT2 | Exostosin glycosyltransferase 2 | GlcNAcα4 & GlcAβ4 |
| (NM_000401) | | transferases: HS copolymerase |
| | | KI: increase GAG production |
| | | KO: decrease GAG production |
| NDST1 | N-deacetylase N-sulfotransferase | N-deacetylation and N-sulfation of GlcNAc |
| (U18918) | 1 | KI: increase N-sulfation |
| | | KO: decrease N-sulfation |
| NDST2 | N-deacetylase N-sulfotransferase | N-deacetylation and N-sulfation of GlcNAc |
| (NM_003635) | 2 | KI: increase N-sulfation |
| | | KO: decrease N-sulfation |
| NDST3 | N-deacetylase N-sulfotransferase | N-deacetylation and N-sulfation of GlcNAc |
| (AF074924) | 3 | KI: increase N-sulfation |
| | | KO: decrease N-sulfation |
| NDST4 | N-deacetylase N-sulfotransferase | N-deacetylation and N-sulfation of GlcNAc |
| (AB036429) | 4 | KI: increase N-sulfation |
| | | KO: decrease N-sulfation |
| C5EPI | C5 glucuronyl epimerase | Conversion of D-GlcA to L-IdoA |
| (XM_035390) | | KI: increase epimerization |
| | | KO: decrease epimerization |
| HS2ST | Heparan sulfate 2-O- | 2-O-sulfation of IdoA |
| (AB024568) | sulfotransferase | KI: increase 2-O-sulfation |
| | | KO: decrease 2-O-sulfation |
| HS6ST1 | Heparan sulfate glucosamine | 6-O-sulfation of glucosamine |
| (AB006179) | 6-O-sulfotransferase 1 | KI: increase 6-O-sulfation |
| | | KO: decrease 6-O-sulfation |
| HS6ST2 | Heparan sulfate glucosamine | 6-O-sulfation of glucosamine |
| (NM_147174) | 6-O-sulfotransferase 2 | KI: increase 6-O-sulfation |
| | | KO: decrease 6-O-sulfation |
| HS6ST2 | Heparan sulfate glucosamine | 6-O-sulfation of glucosamine |
| (NM_147175) | 6-O-sulfotransferase 2 short | KI: increase 6-O-sulfation |
| | | KO: decrease 6-O-sulfation |
| HS6ST3 | Heparan sulfate glucosamine | 6-O-sulfation of glucosamine |
| (AF539426) | 6-O-sulfotransferase 3 | KI: increase 6-O-sulfation |
| | | KO: decrease 6-O-sulfation |
| HS3ST1 | Heparan sulfate glucosamine | 3-O-sulfation of glucosamine |
| (AF019386) | 3-O-sulfotransferase 1 | (GlcAGlcNS(±6S) |
| | | KI: increase 3-O-sulfation |
| | | KO: decrease 3-O-sulfation |
| HS3ST2 | Heparan sulfate glucosamine | 3-O-sulfation of glucosamine in restricted |
| (AF105375) | 3-O-sulfotransferase 2 | domains |
| | | KI: increase 3-O-sulfation |
| | | KO: decrease 3-O-sulfation |
| HS3ST3A1 | Heparan sulfate glucosamine | 3-O-sulfation of glucosamine in restricted |
| (AF105376) | 3-O-sulfotransferase 3A | domains |
| | | KI: increase 3-O-sulfation |
| | | KO: decrease 3-O-sulfation |
| HS3ST3B1 | Heparan sulfate glucosamine | 3-O-sulfation of glucosamine in restricted |
| (AF105377) | 3-O-sulfotransferase 3B | domains |
| | | KI: increase 3-O-sulfation |
| | | KO: decrease 3-O-sulfation |
| HS3ST4 | Heparan sulfate glucosamine | 3-O-sulfation of glucosamine in restricted |
| (AF105378) | 3-O-sulfotransferase 4 | domains |
| HS3ST5 | Heparan sulfate glucosamine | 3-O-sulfation of glucosamine in restricted |
| (AF503292) | 3-O-sulfotransferase 5 | domains |
| | | KI: increase 3-O-sulfation |
| | | KO: decrease 3-O-sulfation |
| HS3ST6 | Heparan sulfate glucosamine | 3-O-sulfation of glucosamine in restricted |
| (AE00640) | 3-O-sulfotransferase 6 | domains |
| | | KI: increase 3-O-sulfation |
| | | KO: decrease 3-O-sulfation |
| SDC-1 | Syndecan-1 | Membrane spanning proteoglycan |
| (HS.82109) | | KI: increase GAG production |
| | | KO: decrease GAG production |
| SDC-2 | Syndecan-2 (fibroglycan) | Membrane spanning proteoglycan |
| (HS.1501) | | KI: increase GAG production |
| | | KO: decrease GAG production |
| SDC-3 | Syndecan-3 (N-syndecan) | Membrane spanning proteoglycan |
| (HS.158287) | | KI: increase GAG production |
| | | KO: decrease GAG production |
| SDC-4 | Syndecan-4 (ryudocan, | Membrane spanning proteoglycan |
| (HS.252189) | amphiglycan) | KI: increase GAG production |
| | | KO: decrease GAG production |
| GPC-1 | Glypican-1 | GPI-anchored membrane proteoglycan |
| (HS.328232) | | KI: increase GAG production |
| | | KO: decrease GAG production |
| GPC-2 | Glypican-2 (cerebroglycan) | GPI-anchored membrane proteoglycan |
| (HS.211701) | | KI: increase GAG production |
| | | KO: decrease GAG production |
| GPC-3 | Glypican-3 | GPI-anchored membrane proteoglycan |
| (HS.119651) | | KI: increase GAG production |
| | | KO: decrease GAG production |
| GPC-4 | Glypican-4 | GPI-anchored membrane proteoglycan |
| (HS.58367) | | KI: increase GAG production |
| | | KO: decrease GAG production |
| GPC-5 | Glypican-5 | GPI-anchored membrane proteoglycan |
| (HS.76828) | | KI: increase GAG production |
| | | KO: decrease GAG production |
| GPC-6 | Glypican-6 | GPI-anchored membrane proteoglycan |
| (HS.118407) | | KI: increase GAG production |
| | | KO: decrease GAG production |
| CD44 | CD44 (epican) | Membrane spanning proteoglycan |
| (HS.502328) | | KI: increase GAG production |
| | | KO: decrease GAG production |
| HSPG2 | Perlecan (HSPG2) | Secreted basement membrane proteoglycan |
| (HS.211573) | | KI: increase GAG production |
| | | KO: decrease GAG production |
| AGRN | Agrin | Secreted basement membrane proteoglycan |
| (HS. 273330) | | KI: increase GAG production |
| | | KO: decrease GAG production |
| SGCN | Serglycin | Intracellular granule proteoglycan |
| (HS.451015) | | KI: increase GAG production |
| | | KO: decrease GAG production |

Additional steps contemplated in methods herein include treating the genetically modified cells to degranulate the cell line. Multiple suitable methods of degranulating cells include but are not limited to methods selected from the group consisting of Antigen-IgE induced FcεRI aggregation on the degranulating cell surface, contacting the cell line to a degranulating agent, altering the culture temperature, altering the culture medium pH, altering the culture medium salt concentration, and agitation. Suitable degranulating agents include but are not limited to calcium ionophore A23187, compound 48/80, tetradecanoyl phorbol acetate (TPA), and substance P.

### Methods of Culturing Stem Cells

Also provided herein are methods of culturing stem cells, comprising culturing the stem cells in a media comprising heparin or heparan sulfate that has been produced in methods disclosed herein. In some cases, methods herein comprise contacting a stem cell to a composition comprising a heparin or a heparan sulfate isolated from a genetically modified cell line. Suitable stem cells for culturing with media comprising heparin and/or heparan sulfate produced using methods herein include but are not limited to hematopoietic stem cells, mesenchymal stem cells, neural stem cells, epithelial stem cells, skin stem cells, embryonic stem cells, and induced pluripotent stem cells.

Heparin and heparan sulfate produced by methods disclosed herein comprises culturing genetically modified cell lines such as at least one of a mastocytoma cell line and a basophil neoplastic cell line. Non-limiting exemplary mastocytoma cell lines for methods herein include MST cells, P815 cells, MC/9 cells, SI/SI4 cells, 10P2 cells, 11P0-1 cells, and 10P12 cells. In some embodiments, the genetically modified cell line is RT4 cells, 682B cells, 751G cells, 1016T cells, KK-47, MGH-U1, MHG-U2, MGH-U3, or MGH-U4. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). Alternately or in addition, the genetically modified cell line is deficient for a heparan sulfate catabolic enzyme. Heparan sulfate catabolic enzyme are contemplated to comprise one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In further embodiments, the genetically modified cell line overexpresses one or more of heparanase, a protease, a polymerase, a sulfotransferase, an endosulfatase, and a proteoglycan protein core. Proteases for methods herein include one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. Suitable proteoglycan protein cores include one or more of serglycin, a syndecan, a glypican, CD44 isoforms (CD44E), perlecan, collagen XVIII, and agrin, and recombinant versions thereof. In some cases, the serglycin comprises a human serglycin, a pig serglycin, a mouse serglycin. Modifications to serglycan include modification of the number of GAG attachment sites in the serglycin. In some cases, the syndecan is selected from the group consisting of syndecan-1, syndecan-2, syndecan-3, syndecan-4, and syndecan ectodomains. Exemplary glypicans are selected from the group consisting of glypican 1, glypican 2, glypican 3, glypican 4, glypican 5, and glypican 6 and ectodomains.

### Methods of Treatment

Therefore, disclosed herein are methods of treating diseases and conditions in subjects in need thereof by administering an effective amount of one or more heparin and/or heparan sulfate purified from mastocytoma or basophil neoplastic cell lines by methods provided herein. Mastocytoma cell lines are contemplated to include MST cells, P815 cells, MC/9 cells, SI/SI4 cells, 10P2 cells, 11P0-1 cells, and 10P12 cells. In some embodiments, the genetically modified cell line is RT4 cells, 682B cells, 751G cells, 1016T cells, KK-47, MGH-U1, MHG-U2, MGH-U3, or MGH-U4. In some embodiments, the disease or condition comprises one or more of wounds, bone fractures, skin conditions, cancers, angiogenesis, inflammatory diseases, neurological diseases, and other suitable diseases.

In some embodiments, the compositions or pharmaceutical compositions disclosed herein are administered to the subject by any suitable route, found to be effective in treating thrombosis, inflammation, cancer, microbial infections, neurodegenerative disorders and wound healing among others. In some embodiments, the compositions or pharmaceutical compositions disclosed herein are administered orally, rectally, sublingually, sublabially, buccally, epidurally, entracerebrally, intracerebroventricalarly, topically, transdermally, nasally, intraarterially, intraarticularly, intracardiacally, intradermally, subcutaneously, intralesionally, intramuscular, intraocularly, intraosseously, intraperitoneally, intrathecally, intravenously, transmucosally, or any other route of administration known by one of skill in the art.

Methods of treatment herein comprise administering an effective amount of a composition comprising one or more heparin and/or heparan sulfate preparations provided herein to a subject in need thereof. In some cases, the method comprises identifying a patient in need of treatment. In some cases, the method comprises monitoring the patient for an improvement in one or more symptoms. In some cases, the method comprises administration of one, two three, four, five, six, seven, eight, nine, ten, or more additional doses of the composition until the subject experiences improvement in one or more symptoms. In some cases, the method comprises continuous or chronic administration of the composition to improve one or more symptoms.

### Methods of Treating Thrombosis

In some embodiments, there is provided a method of treating thrombosis in a subject in need thereof comprising administering to the subject an effective amount of one or more heparin, and/or heparan sulfate prepared from genetically modified mastocytoma cells (e.g. MST cells) or basophil neoplastic cells via methods provided herein. In some embodiments, the thrombosis comprises, venous thrombosis, deep vein thrombosis, portal vein thrombosis, renal vein thrombosis, jugular vein thrombosis, Budd-Chiari syndrome, Paget-Schroetter disease, Cerebral venous sinus thrombosis, Cavernous sinus thrombosis, arterial thrombosis, stroke, myocardial infarction, Hepatic artery thrombosis, acute coronary syndrome atrial fibrillation, or pulmonary embolism. In some embodiments, treatment of the thrombosis reduces swelling, pain, tenderness, skin discoloration, shortness of breath, chest pain, rapid heart rate, cough, or other symptom of thrombosis. In some embodiments, the method prevents or eliminates a blood clot. In some embodiments, the method prevents or eliminates a blood clot without causing heparin-induced thrombocytopenia.

In methods provided herein, treatment of thrombosis with heparin or heparan sulfate compositions produced using methods herein have reduced risk of complications and side effects that often result with treatment using unfractionated heparin. In some cases, heparin or heparan sulfate compositions produced using methods herein useful in methods of treating thrombosis herein have reduced binding to platelet factor 4 (PF4). In some cases, the composition is purified from a mastocytoma (MST) cell line or a basophil neoplastic cell line genetically modified to be deficient for heparan sulfate 2-O-sulfotransferase (HS2ST). In some cases, the composition is purified from a MST cell line or a basophil neoplastic cell line genetically modified to overexpress heparan sulfate-6-O-endosulfatase 1 or 2 (SULF1-2). In some embodiments, heparin compositions herein have reduced platelet factor 4 (PF4) binding while maintaining potent anti-coagulation activity (Factor Xa). In some embodiments, such heparin compositions have reduced side effects. In some embodiments, such heparin compositions have a decrease in 2-O-sulfation. In some embodiments, heparin compositions are prepared from a cell line deficient in 2-O sulfotransferase. In some embodiments, such heparin compositions have a decrease in certain 6-sulfations. In some embodiments, heparin compostions are prepared from a cell line that overexpresses Sulf-2.

Methods of treating thrombosis in a subject in need thereof provided herein comprise administering a composition comprising a heparin or a heparan sulfate isolated from a genetically modified cell line. In some cases, the genetically modified cell line comprises at least one of a mastocytoma (MST) cell line or a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Methods of Treating Wounds

In some embodiments, there is provided a method of treating a wound in a subject in need thereof comprising administering to the subject an effective amount of heparin, and/or heparan sulfate prepared from genetically modified mastocytoma cells (e.g. MST cells) or basophil neoplastic cells via methods provided herein. In some embodiments, the wound comprises an incision, a laceration, an abrasion, an avulsion, a puncture wound, a penetration wound, a gunshot wound, a hematoma, or a crush injury. In some embodiments, the method reduces symptoms or complications related to a wound, such as drainage, pus, fever, or lymph node swelling. In some embodiments, the method speeds the healing time of a wound. In some embodiments, the method treats diabetic wounds. In some embodiments, the method treats a nerve injury. In some embodiments, the method treats a spinal cord injury.

Methods of treating wounds in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Treatment of Inflammation

In some embodiments, there is provided a method of treating inflammation in a subject in need thereof comprising administering to the subject an effective amount of heparin, and/or heparan sulfate prepared from genetically modified mastocytoma cells (e.g. MST cells) or basophil neoplastic cells via methods provided herein. Inflammation herein is contemplated to comprise inflammatory diseases often caused by aberrant immune responses, such as autoimmunity and other diseases associated with an over-active immune response or inappropriate immune response. In some embodiments, the inflammation comprises rheumatoid arthritis, juvenile rheumatoid arthritis, osteoarthritis, psoriatic arthritis, multiple sclerosis (MS), encephalomyelitis, myasthenia gravis, systemic lupus erythematosus (SLE), asthma, allergic asthma, autoimmune thyroiditis, atopic dermatitis, eczematous dermatitis, psoriasis, Sjögren's Syndrome, Crohn's disease, aphthous ulcer, iritis, conjunctivitis, keratoconjunctivitis, ulcerative colitis (UC), inflammatory bowel disease (IBD), cutaneous lupus erythematosus, scleroderma, vaginitis, proctitis, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, interstitial lung fibrosis, chronic obstructive pulmonary disease (COPD), cystic fibrosis, alpha-1 antitrypsin deficiency, acute respiratory distress syndrome (ARDS), Hashimoto's thyroiditis, autoimmune polyglandular syndrome, insulin-dependent diabetes mellitus (IDDM, type I diabetes), insulin-resistant diabetes mellitus (type 2 diabetes), immune-mediated infertility, autoimmune Addison's disease, pemphigus vulgaris, pemphigus foliaceus, dermatitis herpetiformis, autoimmune alopecia, vitiligo, autoimmune hemolytic anemia, autoimmune thrombocytopenic purpura, pernicious anemia, Guillain-Barre syndrome, stiff-man syndrome, acute rheumatic fever, sympathetic ophthalmia, Goodpasture's syndrome, systemic necrotizing vasculitis, antiphospholipid syndrome or an allergy, Behcet's disease, X-linked lymphoproliferative syndrome (SH2D1A/SAP deficiency), hyper IgE syndrome or Graft vs. Host Disease (GVHD). In some embodiments, treatment of the inflammation reduces pain, redness, swelling, loss of joint function, fever, chills, fatigue, headache, loss of appetite, muscle stiffness, or other symptom associated with inflammation or inflammatory disease.

Methods of treating inflammation in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the sulfotransferase is heparan sulfate 2-O-sulfotransferase. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Treatment of Lung Inflammation

In some embodiments, there is provided a method of treating lung inflammation in a subject in need thereof comprising administering to the subject an effective amount of heparin, and/or heparan sulfate prepared from genetically modified mastocytoma cells (e.g. MST cells) or basophil neoplastic cells via methods provided herein. Lung inflammation herein is contemplated to comprise inflammatory diseases of the lung often caused by aberrant immune responses, such as autoimmunity and other diseases associated with an over-active immune response or inappropriate immune response. In some embodiments, the lung inflammation comprises asthma, allergic asthma, interstitial lung fibrosis, chronic obstructive pulmonary disease (COPD), cystic fibrosis, alpha-1 antitrypsin deficiency, or acute respiratory distress syndrome (ARDS). In some embodiments, treatment of the inflammation improves breathing and blood oxygenation, reduces incidence of shortness of breath, wheezing, chest tightness, cough, and respiratory infections. In some cases, the treatment reduces need for supplemental oxygen, use of rescue inhalers or steroid medication.

Methods of treating inflammation in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the sulfotransferase is heparan sulfate 2-O-sulfotransferase. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Treatment of Cancer

In some embodiments, there is provided a method of treating cancer in a subject in need thereof comprising administering to the subject an effective amount of heparin, and/or heparan sulfate prepared from genetically modified mastocytoma cells (e.g. MST cells) or basophil neoplastic cells via methods provided herein. In some embodiments, the cancer comprises Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute mycloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Vermcous carcinoma, Visual Pathway Glioma, Vulvar Cancer, Waldenstrom's macroglobulinemia, Warthin's tumor, Wilms' tumor, or other type of cancer. In some embodiments, the cancer comprises a metastasis of one or more of the above cancers.

Methods of treating cancer in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

Efficacy in treating cancer in particular may be measured by any suitable metric. In some embodiments, therapeutic efficacy is measured based on an effect of treating a proliferative disorder, such as cancer. In general, therapeutic efficacy of the methods and compositions of the invention, with regard to the treatment of a proliferative disorder (e.g. cancer, whether benign or malignant), may be measured by the degree to which the methods and compositions promote inhibition of tumor cell proliferation, the inhibition of tumor vascularization, the eradication of tumor cells, and/or a reduction in the size of at least one tumor such that a human is treated for the proliferative disorder. Several parameters to be considered in the determination of therapeutic efficacy are discussed herein. The proper combination of parameters for a particular situation can be established by the clinician. The progress of the inventive method in treating cancer (e.g., reducing tumor size or eradicating cancerous cells) can be ascertained using any suitable method, such as those methods currently used in the clinic to track tumor size and cancer progress. In some embodiments, the primary efficacy parameter used to evaluate the treatment of cancer preferably is a reduction in the size of a tumor. Tumor size can be determined using any suitable technique, such as measurement of dimensions, or estimation of tumor volume using available computer software, such as FreeFlight software developed at Wake Forest University that enables accurate estimation of tumor volume. Tumor size can be determined by tumor visualization using, for example, CT, ultrasound, SPECT, spiral CT, MRI, photographs, and the like. In embodiments where a tumor is surgically resected after completion of the therapeutic period, the presence of tumor tissue and tumor size can be determined by gross analysis of the tissue to be resected, and/or by pathological analysis of the resected tissue.

Desirably, the growth of a tumor is stabilized (i.e., one or more tumors do not increase more than 1%, 5%, 10%, 15%, or 20% in size, and/or do not metastasize) as a result of treatment. In some embodiments, a tumor is stabilized for at least about 1, 2, 3, 4, 5, 6, 7, 8, 9, 10, 11, 12, or more weeks. In some embodiments, a tumor is stabilized for at least about 1, 2, 3, 4, 5, 6, 7, 8, 9, 10, 11, 12, or more months. In some embodiments, a tumor is stabilized for at least about 1, 2, 3, 4, 5, 6, 7, 8, 9, 10, or more years. In some embodiments, the size of a tumor is reduced at least about 5% (e.g., at least about 10%, 15%, 20%, or 25%). In some embodiments, tumor size is reduced at least about 30% (e.g., at least about 35%, 40%, 45%, 50%, 55%, 60%, or 65%). In some embodiments, tumor size is reduced at least about 70% (e.g., at least about 75%, 80%, 85%, 90%, or 95%). In some embodiments, the tumor is completely eliminated, or reduced below a level of detection. In some embodiments, a subject remains tumor free (e.g. in remission) for at least about 1, 2, 3, 4, 5, 6, 7, 8, 9, 10, 11, 12, or more weeks following treatment. In some embodiments, a subject remains tumor free for at least about 1, 2, 3, 4, 5, 6, 7, 8, 9, 10, 11, 12, or more months following treatment. In some embodiments, a subject remains tumor free for at least about 1, 2, 3, 4, 5, 6, 7, 8, 9, 10, or more years after treatment.

When a tumor is subject to surgical resection following completion of the therapeutic period, the efficacy of the inventive method in reducing tumor size can be determined by measuring the percentage of resected tissue that is necrotic (i.e., dead). In some embodiments, a treatment is therapeutically effective if the necrosis percentage of the resected tissue is greater than about 20% (e.g., at least about 30%, 40%, 50%, 60%, 70%, 80%, 90%, or 100%), more preferably about 90% or greater (e.g., about 90%, 95%, or 100%). Most preferably, the necrosis percentage of the resected tissue is about 100%, that is, no tumor tissue is present or detectable.

A number of secondary parameters can be employed to determine the efficacy of the inventive method. Examples of secondary parameters include, but are not limited to, detection of new tumors, detection of tumor antigens or markers (e.g., CEA, PSA, or CA-125), biopsy, surgical downstaging (i.e., conversion of the surgical stage of a tumor from unresectable to resectable), PET scans, survival, disease progression-free survival, time to disease progression, quality of life assessments such as the Clinical Benefit Response Assessment, and the like, all of which can point to the overall progression (or regression) of cancer in a human. Biopsy is particularly useful in detecting the eradication of cancerous cells within a tissue. Radioimmunodetection (RAID) is used to locate and stage tumors using serum levels of markers (antigens) produced by and/or associated with tumors (“tumor markers” or “tumor-associated antigens”), and can be useful as a pre-treatment diagnostic predicate, a post-treatment diagnostic indicator of recurrence, and a post-treatment indicator of therapeutic efficacy. Examples of tumor markers or tumor-associated antigens that can be evaluated as indicators of therapeutic efficacy include, but are not limited to, carcinembryonic antigen (CEA) prostate-specific antigen (PSA), CA-125, CA19-9, ganglioside molecules (e.g., GM2, GD2, and GD3), MART-1, heat shock proteins (e.g., gp96), sialyl Tn (STn), tyrosinase, MUC-1, HER-2/neu, c-erb-B2, KSA, PSMA, p53, RAS, EGF-R, VEGF, MAGE, and gp100. Other tumor-associated antigens are known in the art. RAID technology in combination with endoscopic detection systems also efficiently distinguishes small tumors from surrounding tissue.

In some embodiments, the treatment of cancer in a human patient is evidenced by one or more of the following results: (a) the complete disappearance of a tumor (i.e., a complete response), (b) about a 25% to about a 50% reduction in the size of a tumor for at least four weeks after completion of the therapeutic period as compared to the size of the tumor before treatment, (c) at least about a 50% reduction in the size of a tumor for at least four weeks after completion of the therapeutic period as compared to the size of the tumor before the therapeutic period, and (d) at least a 2% decrease (e.g., about a 5%, 10%, 20%, 30%, 40%, 50%, 60%, 70%, 80% or 90% decrease) in a specific tumor-associated antigen level at about 4-12 weeks after completion of the therapeutic period as compared to the tumor-associated antigen level before the therapeutic period. While at least a 2% decrease in a tumor-associated antigen level is preferred, any decrease in the tumor-associated antigen level is evidence of treatment of a cancer in a patient. For example, with respect to unresectable, locally advanced pancreatic cancer, treatment can be evidenced by at least a 10% decrease in the CA19-9 tumor-associated antigen level at 4-12 weeks after completion of the therapeutic period as compared to the CA19-9 level before the therapeutic period. Similarly, with respect to locally advanced rectal cancer, treatment can be evidenced by at least a 10% decrease in the CEA tumor-associated antigen level at 4-12 weeks after completion of the therapeutic period as compared to the CEA level before the therapeutic period.

With respect to quality of life assessments, such as the Clinical Benefit Response Criteria, the therapeutic benefit of the treatment in accordance with the invention can be evidenced in terms of pain intensity, analgesic consumption, and/or the Karnofsky Performance Scale score. The Karnofsky Performance Scale allows patients to be classified according to their functional impairment. The Karnofsky Performance Scale is scored from 0-100. In general, a lower Karnofsky score is predictive of a poor prognosis for survival. Thus, the treatment of cancer in a human patient alternatively, or in addition, is evidenced by (a) at least a 50% decrease (e.g., at least a 60%, 70%, 80%, 90%, or 100% decrease) in pain intensity reported by a patient, such as for any consecutive four week period in the 12 weeks after completion of treatment, as compared to the pain intensity reported by the patient before treatment, (b) at least a 50% decrease (e.g., at least a 60%, 70%, 80%, 90%, or 100% decrease) in analgesic consumption reported by a patient, such as for any consecutive four week period in the 12 weeks after completion of treatment as compared to the analgesic consumption reported by the patient before treatment, and/or (c) at least a 20 point increase (e.g., at least a 30 point, 50 point, 70 point, or 90 point increase) in the Kamofsky Performance Scale score reported by a patient, such as for any consecutive four week period in the 12 weeks after completion of the therapeutic period as compared to the Kamofsky Performance Scale score reported by the patient before the therapeutic period.

The treatment of a proliferative disorder (e.g. cancer, whether benign or malignant) in a human patient desirably is evidenced by one or more (in any combination) of the foregoing results, although alternative or additional results of the referenced tests and/or other tests can evidence treatment efficacy.

In some embodiments, tumor size is reduced preferably without significant adverse events in the subject. Adverse events are categorized or “graded” by the Cancer Therapy Evaluation Program (CTEP) of the National Cancer Institute (NCI), with Grade 0 representing minimal adverse side effects and Grade 4 representing the most severe adverse events. The NCI toxicity scale (published April 1999) and Common Toxicity Criteria Manual (updated August 1999) is available through the NCI, e.g., or in the Investigator's Handbook for participants in clinical trials of investigational agents sponsored by the Division of Cancer Treatment and Diagnosis, NCI (updated March 1998). Desirably, methods described herein are associated with minimal adverse events, e.g. Grade 0, Grade 1, or Grade 2 adverse events, as graded by the CTEP/NCI. However, reduction of tumor size, although preferred, is not required in that the actual size of tumor may not shrink despite the eradication (such as in necrosis) of tumor cells. Eradication of cancerous cells is sufficient to realize a therapeutic effect. Likewise, any reduction in tumor size is sufficient to realize a therapeutic effect.

Detection, monitoring, and rating of various cancers in a human are further described in Cancer Facts and FIGS. 2001, American Cancer Society, New York, N.Y. Accordingly, a clinician can use standard tests to determine the efficacy of the various embodiments of the inventive method in treating cancer. However, in addition to tumor size and spread, the clinician also may consider quality of life and survival of the patient in evaluating efficacy of treatment.

### Treatment of Neurodegenerative Disorders

In some embodiments, there is provided a method of treating a neurodegenerative disorder in a subject in need thereof comprising administering to the subject an effective amount of heparin, and/or heparan sulfate prepared from genetically modified mastocytoma cells (e.g. MST cells) or basophil neoplastic cells via methods provided herein. In some embodiments, the neurodegenerative disorder comprises Alzheimer's disease, Parkinson's disease, Huntington's disease, Amyotrophic lateral sclerosis, Dementia, Transmissible spongiform encephalopathy, Dentatorubro-pallidoluysian atrophy, Spinal and bulbar muscular atrophy, Spinocerebellar ataxia Type 1, Spinocerebellar ataxia Type 2, Spinocerebellar ataxia Type 3, Spinocerebellar ataxia Type 6, Spinocerebellar ataxia Type 7, or Spinocerebellar ataxia Type 17. In some embodiments, the method reduces symptoms of a neurodegenerative disorder such as memory loss, disorientation, confusion, mood and/or personality disorder, tremor, bradykinesia, muscle rigidity, balance impairment, speech disorder, choria, dystonia, ataxia, swallowing disorder, irritability, sadness, apathy, social withdrawal, insomnia, fatigue, suicidal thoughts, weakness, speech disorder, muscle cramping, impaired coordination, stumbling, unsteady gait, uncontrolled movements, slurred speech, vocal changes, or headache. In some embodiments, the method delays onset of more severe symptoms. In some embodiments, the delay is 1, 2, 3, 4, 5, 6 or more weeks, 1, 2, 3, 4, 5, 6, 7, 8, 9, 10, 11, 12, or more months, 1, 2, 3, 4, 5, 6, 7, 8, 9, 10, 11, 12, 13, 14, 15, 20, 25, 30, or more years.

Methods of treating neurodegenerative disorders in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Methods of Treating Bone Fractures

In some embodiments, there is provided a method of treating a bone fracture in a subject in need thereof comprising administering to the subject an effective amount of heparin, and/or heparan sulfate prepared from genetically modified mastocytoma cells (e.g. MST cells) or basophil neoplastic cells via methods provided herein. Bone fractures suitable for treatment via methods provided herein include but are not limited to stress fractures, pathologic fractures, oblique fractures, transverse fractures, comminuted fractures, greenstick fractures, buckle fractures, growth plate fractures, and other suitable fractures. In some cases, treatment of bone fractures herein speeds healing of fractures by at least about 1 week, 2 weeks, 3, weeks, 4 weeks, 5 weeks, or more. In some cases, treatment of bone fractures herein eliminates need for surgery to correct the fracture.

Methods of treating thrombosis in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Methods of Dermatological Treatment

In some embodiments, there is provided a method of treating a dermatological condition in a subject in need thereof comprising administering to the subject an effective amount of heparin, and/or heparan sulfate prepared from genetically modified mastocytoma cells (e.g. MST cells) or basophil neoplastic cells via methods provided herein. Dermatological conditions suitable for treatment via methods provided herein include but are not limited to a wrinkle, a pimple, a hyperpigmentation, an age-related skin condition, dryness, lack of skin elasticity, lack of skin firmness, and fine lines. In further embodiments, the skin condition is fine lines and wrinkles; age spots and dyspigmentation; skin texture, tone and elasticity; roughness and photo damage; the ability of skin to regenerate itself; environmental damage; smoothness and tightness skin; age spots, fine and coarse lines and wrinkles, fine and course periocular wrinkles, nasolabial folds, facial fine and coarse lines, skin radiance and evenness, skin firmness, hyperpigmentation, dark spots and/or patches, skin brightness and youthful appearance, photo aged skin, intrinsically and extrinsically aged skin, skin cellular turnover, skin barrier, skin's ability to retain moisture, brown and red blotchiness, redness, skin epidermal thickness, dermal epidermal junction, pore size and number of pores, or a combination thereof. In some cases, the methods rejuvenate sun damaged and aging skin; improves the appearance of fine lines and wrinkles; promotes cell renewal; diminishes the appearance of age spots and dyspigmentation; improves skin tone, texture and elasticity; reduces roughness and photo damage; prevents or reduces environmental damage; plumps the skin; brightens the skin; lightens the skin; strengthens the ability of skin to regenerate itself; improves the appearance of age spots; brightens and lightens age spots; improves skin firmness, elasticity, resiliency; smooths, tightens, or fills in fine lines on the skin; reduces the appearance of dark circles under the eye; improves lip texture or condition; enhances natural lip color; increases lip volume; promotes epithelialization of post-procedure skin; restores the skin's barrier or moisture balance; improves the appearance of age spots; improves the appearance of skin pigmentation, or a combination thereof. In one embodiment, the compositions reduce the appearance of fine lines and wrinkles; diminish the appearance of age spots and dyspigmentation; improve skin texture, tone and elasticity; reduce roughness and photo damage; strengthen the ability of skin to regenerate itself; prevent or reduce environmental damage; smooth and tightens skin; brighten and lighten age spots, reduce in fine and coarse lines and wrinkles, improve appearance of fine and course periocular wrinkles, improve appearance of nasolabial folds, improve perioral wrinkles, improve facial fine and coarse lines, improve skin tone, radiance and evenness, improve skin firmness, reduce tactile roughness, improve skin texture, overall photo damage, overall hyperpigmentation, global improvement, reduce in appearance of dark spots and/or patches, improve appearance of skin brightness and youthful appearance, improve overall condition of skin, improve the appearance of photo aged skin, improve appearance of intrinsically and extrinsically aged skin, improve skin cellular turnover, improve skin barrier, improve skin's ability to retain moisture, reduce the appearance of brown and red blotchiness, redness, increase skin epidermal thickness, strengthen dermal epidermal junction, reduce the appearance of pore size and pores, improve smoothness, or a combination thereof.

Methods of treating a skin condition in a subject in need thereof provided herein comprise administering a composition comprising a heparin or a heparan sulfate isolated from a genetically modified cell line. In some cases, the genetically modified cell line comprises at least one of a mastocytoma (MST) cell line and a basophil neoplastic cell line. In some cases, the genetically modified cell line is deficient for one or more of chondroitin sulfate synthase 1 (CHSY1), chondroitin sulfate N-acetylgalactosaminyltransferase 2 (CSGALNACT2), chondroitin sulfate N-acetylgalactosaminyltransferase 1 (CSGALNACT1), Heparan sulfate 2-O-sulfotransferase (HS2ST), and heparan sulfate C5-epimerase (GLCE). In some cases, the genetically modified cell line is deficient for a heparan sulfate catabolic enzyme. In some cases, the heparan sulfate catabolic enzyme comprises one or more of heparanase (HPSE), beta-glucoronidase (GUSB), sulfamidase (SGSH), heparan alpha-glucosaminide N-acetyltransferase (HGNAT), alpha-N-acetyl glucosaminidase (GSNAT), uronate-2-O-sulfatases (IDS and GDS), alpha-L-iduronidase (IDUA), and heparan sulfate 6-O-endosulfatase (SULF1-2). In some cases, the genetically modified MST cell line overexpresses one or more of heparanase, a protease, a polymerase, a sulfotransferase, an endosulfatase, and a proteoglycan protein core. In some cases, the protease comprises one or more of matrix metalloproteinase (MMP), MMP2, MMP3, MMP7, MMP9, MT1-MMP, MT3-MMP, ADAM17, ADAMTS1, ADAMTS4, trypsin, and chymotrypsin. In some cases, the proteoglycan protein core comprises one or more of serglycin, a syndecan, a glypican, perlecan, and CD44 core protein (CD44E). In some cases, the serglycin comprises a human serglycin, a pig serglycin, a mouse serglycin. In some cases, the number of GAG attachment sites in the serglycin is modified. In some cases, the syndecan is selected from the group consisting of syndecan-1, syndecan-2, syndecan-3, syndecan-4, and syndecan ectodomains. In some cases, the glypican is selected from the group consisting of glypican 1, glypican 2, glypican 3, glypican 4, glypican 5, and glypican 6 and ectodomains.

### Heparin and Heparan Sulfate Compositions

Provided herein are compositions comprising heparin or heparan sulfate having reduced affinity for a platelet factor compared to unfractionated heparin. In some cases, the platelet factor is a platelet factor 4 (PF4). Such compositions herein often have a lower risk of complications often observed in subjects treated with unfractionated heparin. Heparin and heparan sulfate compositions herein are often isolated from genetically modified cell lines, such as mastocytoma cell lines or basophil neoplastic cell lines. In some cases, the genetically modified cell lines are selected from the group consisting of MST cells, P815 cells, MC/9 cells, SI/SI4 cells, 10P2 cells, 11P0-1 cells, and 10P12 cells. In some cases, the composition is purified from a mastocytoma (MST) cell line or a basophil neoplastic cell line genetically modified to be deficient for Heparan sulfate 2-O-sulfotransferase (HS2ST). In some embodiments, the genetically modified cell line is RT4 cells, 682B cells, 751G cells, 1016T cells, KK-47, MGH-U1, MHG-U2, MGH-U3, or MGH-U4. In some cases, the composition is purified from a MST cell line or a basophil neoplastic cell line genetically modified to overexpress Heparan sulfate-6-O-endosulfatase 1 and 2 (SULF1-2). In some embodiments, heparin compositions herein have reduced platelet factor 4 (PF4) binding while maintaining potent anti-coagulation activity (Factor Xa). In some embodiments, such heparin compositions have reduced side effects. In some embodiments, such heparin compositions have a decrease in 2-O-sulfation. In some embodiments, heparin compositions are prepared from a cell line deficient in 2-O sulfotransferase. In some embodiments, such heparin compositions have a decrease in certain 6-sulfations. In some embodiments, heparin compostions are prepared from a cell line that overexpresses Sulf-2.

### High-Throughput Methods of Measuring Heparin and Heparan Sulfate

Also provided herein are high throughput methods of quantifying heparan sulfate in a group of samples. High throughput analysis of heparin and heparan sulfate samples is valuable in optimizing methods of production of heparin and heparan sulfate disclosed herein. In some embodiments, the methods comprise: (a) binding each sample to a well of a multi-well chromatography column; (b) digesting the samples bound to the column with an enzyme; (c) eluting the samples from the column with a solution comprising a salt; and (d) measuring the heparan sulfate in the sample using liquid chromatography. In some embodiments, the chromatography column is selected from at least one of an ion exchange column and a size exclusion column. In some embodiments, the enzyme is selected from at least one of a nuclease and a protease. In some embodiments, the salt is a volatile salt. In some embodiments, the liquid chromatography is an ultra performance liquid chromatography. In some embodiments, the method comprises liquid chromatography with fluorescently tagged heparan sulfate disaccharides.

Further provided herein are high throughput methods of quantifying heparan sulfate in a group of samples, the methods comprising: (a) contacting each sample to a well in a multi-well plate, wherein each well is coated with a guanidinylated antibiotic, thereby binding the heparan sulfate in the sample to the plate; (b) contacting the bound heparan sulfate to a heparan sulfate binding protein; (c) contacting the bound heparan sulfate binding protein to a detection reagent; and (d) measuring a signal from the detection reagent, wherein the signal from the detection reagent corresponds to the amount of heparan sulfate in the sample. In some embodiments, the guanidinylated antibiotic comprises guanidinylated neomycin. In some embodiments, the heparan sulfate binding protein is selected from at least one of FGF-2, PF4, ATIII, and VEGF. In some embodiments, the signal is selected from at least one of a fluorescent signal; a luminescent signal; and a colorimetric signal. In some embodiments, the signal is generated enzymatically.

Suitable heparin and heparan sulfate binding proteins for detection of heparin and heparan sulfate include but are not limited to 4F2 cell-surface antigen heavy chain (4F2hc); 5′-nucleotidase (5′-NT); Alpha-1-antitrypsin (Alpha-1 protease inhibitor); Alpha-1B-glycoprotein (Alpha-1-B glycoprotein); Alpha-2-macroglobulin (Alpha-2-M); Amyloid beta A4 protein (ABPP); Alpha-1-antichymotrypsin (ACT); Angio-associated migratory cell protein; Bile salt export pump (ATP-binding cassette sub-family B member 11); ATP-binding cassette sub-family G member 2 (CD antigen CD338); ATP-binding cassette sub-family G member 5 (Sterolin-1); Amiloride-sensitive amine oxidase [copper-containing](DAO) (Diamine oxidase); Alpha-1B adrenergic receptor (Alpha-1B adrenoreceptor); Agouti-related protein; Aminoacyl tRNA synthase complex-interacting multifunctional protein 1 (Multisynthase complex auxiliary component p43); Aldose reductase (AR); Protein AMBP; Inter-alpha-trypsin inhibitor light chain (ITI-LC); Alpha-2-macroglobulin receptor-associated protein (Alpha-2-MRAP); Angiogenin (RNase 5); Angiotensinogen (Serpin A8); Antithrombin-III (ATIII); Annexin A1; Annexin A2; Annexin A3; Annexin A5; Annexin A6; Amyloid-like protein 1 (APLP-1); Amyloid-like protein 2 (APLP-2); Apolipoprotein A-V (Apo-AV); Apolipoprotein B-100 (Apo B-100); Apolipoprotein E (Apo-E); Beta-2-glycoprotein 1 (Beta(2)GPI); Aquaporin-1 (AQP-1); Arginase-1; Artemin; Agouti-signaling protein (ASP); Sodium/potassium-transporting ATPase subunit alpha-1 (Na(+)/K(+) ATPase alpha-1 subunit); Sodium/potassium-transporting ATPase subunit beta-1 (Sodium/potassium-dependent ATPase subunit beta-1); Sodium/potassium-transporting ATPase subunit beta-3 (Sodium/potassium-dependent ATPase subunit beta-3); Plasma membrane calcium-transporting ATPase 1 (PMCA1); Copper-transporting ATPase 2; ATP synthase subunit alpha, mitochondrial; Attractin (DPPT-L); A disintegrin and metalloproteinase with thrombospondin motifs 1 (ADAM-TS 1); A disintegrin and metalloproteinase with thrombospondin motifs 3 (ADAM-TS 3); A disintegrin and metalloproteinase with thrombospondin motifs 5 (ADAM-TS 5); A disintegrin and metalloproteinase with thrombospondin motifs 8 (ADAM-TS 8); A disintegrin and metalloproteinase with thrombospondin motifs 9 (ADAM-TS 9); Beta-2-microglobulin; Band 3 anion transport protein (Anion exchange protein 1); cDNA FLJ57339, highly similar to Complement C3; Beta-secretase 1; Bone morphogenetic protein 2 (BMP-2); Bone morphogenetic protein 3 (BMP-3); Bone morphogenetic protein 4 (BMP-4); Bone morphogenetic protein 6 (BMP-6); Bone morphogenetic protein 7 (BMP-7); Probetacellulin; Complement C1q subcomponent subunit A; Complement C1q subcomponent subunit B; Complement C1q subcomponent subunit C; C4b-binding protein alpha chain (C4 bp); Voltage-dependent L-type calcium channel subunit alpha-1S; Cadherin-8; Azurocidin (Heparin-binding protein); Cathepsin B; Cathepsin G; Corticosteroid-binding globulin; Carboxypeptidase B2; Carboxypeptidase D; Coiled-coil domain-containing protein 134; Coiled-coil domain-containing protein 80; C-C motif chemokine 1; Eotaxin (C-C motif chemokine 11); C-C motif chemokine 13 (CK-beta-10); C-C motif chemokine 15 (Chemokine CC-2); C-C motif chemokine 17 (CC chemokine TARC); C-C motif chemokine 19; C-C motif chemokine 2 (HC11); C-C motif chemokine 21 (6Ckine); C-C motif chemokine 22 (CC chemokine STCP-1); C-C motif chemokine 23 (CK-beta-8); C-C motif chemokine 24 (CK-beta-6); C-C motif chemokine 25 (Chemokine TECK); C-C motif chemokine 27 (CC chemokine ILC); C-C motif chemokine 28 (Mucosae-associated epithelial chemokine); C-C motif chemokine 3; C-C motif chemokine 4; C-C motif chemokine 5 (EoCP); C-C motif chemokine 7 (Monocyte chemoattractant protein 3); C-C motif chemokine 8 (HC14); Fibronectin type-III domain-containing protein C4orf31; Antigen-presenting glycoprotein CD1d (R3G1) (CD antigen CD1d); Platelet glycoprotein 4; Leukocyte surface antigen CD47; Bile salt-activated lipase (BAL); Ceruloplasmin (Ferroxidase); Uncharacterized protein C6orf15 (Protein STG); Complement factor B; Complement factor D; Complement factor H (H factor 1); Complement factor I; Chordin; UPF0765 protein C10orf58; Clusterin; Chymase; Collagen alpha-1(I) chain (Alpha-1 type I collagen); Collagen alpha-2(I) chain (Alpha-2 type I collagen); Complement C2; Collagen alpha-1(II) chain; Complement C3; Collagen alpha-1(III) chain; Complement C4-A (Acidic complement C4); Collagen alpha-1(IV) chain; Collagen alpha-2(IV) chain; Complement C5; Collagen alpha-1(V) chain; Collagen alpha-3(V) chain; Complement component C6; Collagen alpha-3(VI) chain; Complement component C7; Complement component C8 alpha chain; Complement component C8 beta chain (Complement component 8 subunit beta); Complement component C8 gamma chain; Complement component C9; Collagen alpha-1(IX) chain; Collagen alpha-1(XI) chain; Collagen alpha-2(XI) chain; Collagen alpha-1(XII) chain; Collagen alpha-1(XIII) chain (COLXIIIA1); Collagen alpha-1(XIV) chain (Undulin); Collagen alpha-1(XVIII) chain; Collagen alpha-1(XIX) chain (Collagen alpha-1(Y) chain); Acetylcholinesterase collagenic tail peptide (AChE Q subunit); Cartilage oligomeric matrix protein (COMP) (Thrombospondin-5) (TSP5); Catechol O-methyltransferase; Collagen alpha-1(XXIII) chain; Collagen alpha-1(XXV) chain; Calcium release-activated calcium channel protein 1; Cysteine-rich secretory protein LCCL domain-containing 2; Granulocyte-macrophage colony-stimulating factor (GM-CSF); Connective tissue growth factor; Low affinity cationic amino acid transporter 2 (CAT-2); Gap junction beta-1 protein (Connexin-32); C-X-C motif chemokine 2; C-X-C motif chemokine 6 (Chemokine alpha 3); Platelet basic protein (PBP); C-X-C motif chemokine 10; C-X-C motif chemokine 11; C-X-C motif chemokine 13; C-X-C motif chemokine 16; Cytochrome c; Protein CYR61; Netrin receptor DCC; Estradiol 17-beta-dehydrogenase 11; Estradiol 17-beta-dehydrogenase 12; 17-beta-hydroxysteroid dehydrogenase 13; 3-keto-steroid reductase; Dipeptidyl peptidase 4; Endothelin-converting enzyme 1; Extracellular matrix protein 2; Ephrin-A1 (EPH-related receptor tyrosine kinase ligand 1); Ephrin-A3 (EFL-2); Ephrin-A5 (AL-1); Elastin (Tropoelastin); Neutrophil elastase; Alpha-enolase; Ectonucleotide pyrophosphatase/phosphodiesterase family member 1 (E-NPP 1); Ectonucleotide pyrophosphatase/phosphodiesterase family member 3 (E-NPP 3); Receptor tyrosine-protein kinase erbB-2; Coagulation factor X; Coagulation factor XI; Coagulation factor XII; Protein FAM55A; Coagulation factor IX; Fibulin-7 (FIBL-7); Fibrillin-1; Fibrillin-2; Fibrosin-1; IgG receptor FcRn large subunit p51 (FcRn); Fetuin-B; Heparin-binding growth factor 1 (HBGF-1); Fibroblast growth factor 10 (FGF-10); Fibroblast growth factor 12 (FGF-12); Fibroblast growth factor 14 (FGF-14); Fibroblast growth factor 16 (FGF-16); Fibroblast growth factor 17 (FGF-17); Fibroblast growth factor 18 (FGF-18); Heparin-binding growth factor 2 (HBGF-2) (bFGF); Fibroblast growth factor 2 (FGF-2); Fibroblast growth factor 20 (FGF-20); Fibroblast growth factor 22 (FGF-22); Fibroblast growth factor 3 (FGF-3); Fibroblast growth factor 4 (FGF-4); Fibroblast growth factor 5 (FGF-5); Fibroblast growth factor 6 (FGF-6); Keratinocyte growth factor (KGF); Fibroblast growth factor 8 (FGF-8); Glia-activating factor (GAF); Fibroblast growth factor-binding protein 1 (FGF-BP); Fibroblast growth factor-binding protein 3 (FGF-BP3); Basic fibroblast growth factor receptor 1 (FGFR-1); Fibroblast growth factor receptor 2 (FGFR-2); Fibroblast growth factor receptor 3 (FGFR-3); Fibroblast growth factor receptor 4 (FGFR-4); Fibrinogen alpha chain; Fibrinogen beta chain; Fibrinogen gamma chain; Fibronectin (FN); Follistatin (FS); Follistatin-related protein 1; Furin; Protein G6b; Glia-derived nexin (GDN); Glial cell line-derived neurotrophic factor (hGDNF); Gelsolin (AGEL); Growth hormone receptor (GH receptor); G-protein coupled receptor 182; Transmembrane glycoprotein NMB (Transmembrane glycoprotein HGFIN); Growth-regulated alpha protein (C-X-C motif chemokine 1); Solute carrier family 2, facilitated glucose transporter member 2 (GLUT-2); Proheparin-binding EGF-like growth factor; Hepatoma-derived growth factor (HDGF); Heparin cofactor 2 (Heparin cofactor II) (HC-II); Hereditary hemochromatosis protein (HLA-H); Hepatocyte growth factor (Hepatopoeitin-A); High mobility group protein B1 (HMG-1); Haptoglobin; Histidine-rich glycoprotein (HPRG); Islet amyloid polypeptide (Amylin); Insulin-like growth factor-binding protein 2 (IBP-2); Insulin-like growth factor-binding protein 3 (IBP-3); Insulin-like growth factor-binding protein 4 (IBP-4); Insulin-like growth factor-binding protein 5 (IBP-5); Insulin-like growth factor-binding protein 6 (IBP-6); Plasma protease C1 inhibitor (C1 Inh); Interferon gamma (IFN-gamma); Indian hedgehog protein (IHH); Interferon-inducible GTPase 5; Interleukin-10 (IL-10); Interleukin-12 subunit beta (IL-12B); Interleukin-2 (IL-2); Interleukin-3 (IL-3); Interleukin-4 (IL-4); Interleukin-5 (IL-5); Interleukin-6 (IL-6); Interleukin-7 (IL-7); Interleukin-8 (IL-8); Interphotoreceptor matrix proteoglycan 2; Inhibin beta A chain; Insulin receptor (IR); Plasma serine protease inhibitor; Integrin alpha-1; Integrin alpha-5; Integrin alpha-M; Integrin alpha-V; Integrin beta-1; Integrin beta-3; Inter-alpha-trypsin inhibitor heavy chain H3; Integral membrane protein 2B; Anosmin-1; Putative keratinocyte growth factor-like protein 1; Putative keratinocyte growth factor-like protein 2; Kininogen-1 (Alpha-2-thiol proteinase inhibitor); Laminin subunit alpha-1 (Laminin A chain); Laminin subunit alpha-2 (Laminin M chain); Laminin subunit alpha-3; Laminin subunit alpha-4; Laminin subunit alpha-5; Laminin subunit gamma-2; Leucyl-cystinyl aminopeptidase; Low-density lipoprotein receptor (LDL receptor); Galectin-9 (Gal-9); Leucine-rich repeat-containing G-protein coupled receptor 4 (G-protein coupled receptor 48); Leukemia inhibitory factor receptor (LIF receptor); Hepatic triacylglycerol lipase (HL); Endothelial lipase; Lipoprotein lipase (LPL); Platelet-activating factor acetylhydrolase IB subunit alpha (Lissencephaly-1 protein); Latrophilin-2; Latent-transforming growth factor beta-binding protein 1 (LTBP-1); L-selectin; P-selectin; Mannose-binding protein C (MBP-C); Multidrug resistance protein 1; Multidrug resistance protein 3; Hepatocyte growth factor receptor (HGF receptor); Macrophage migration inhibitory factor (MIF); Midkine (MK); Matrix metalloproteinase-14 (MMP-14); 72 kDa type IV collagenase; Matrilysin; Matrix metalloproteinase-9 (MMP-9); Monocarboxylate transporter 1 (MCT 1); Monocarboxylate transporter 8 (MCT 8); Multidrug resistance-associated protein 6 (ATP-binding cassette sub-family C member 6); Myosin regulatory light polypeptide 9; Neuron navigator 2; Neural cell adhesion molecule 1 (N-CAM-1); Netrin-1; Nicastrin; Noggin; Pro-neuregulin-1; Neuropilin-1; Neurturin; Sodium/bile acid cotransporter; Occludin; Zinc finger protein OZF; Calcium-dependent phospholipase A2; Phospholipase A2, membrane associated; Plasminogen activator inhibitor 1 (PAI); Plasminogen activator inhibitor 1 RNA-binding protein (PAI1 RNA-binding protein 1); Proton-coupled folate transporter (G21); Procollagen C-endopeptidase enhancer 2; Proprotein convertase subtilisin/kexin type 5; Proprotein convertase subtilisin/kexin type 6; Programmed cell death protein 5; Platelet-derived growth factor subunit A (PDGF subunit A); Platelet-derived growth factor subunit B (PDGF subunit B); Protein disulfide-isomerase (PDI); Protein disulfide-isomerase A6; Phosphatidylethanolamine-binding protein 1 (PEBP-1); Platelet endothelial cell adhesion molecule (PECAM-1); Pigment epithelium-derived factor (PEDF); Myeloperoxidase (MPO); Platelet factor 4 variant; Basement membrane-specific heparan sulfate proteoglycan core protein (Perlecan); Biglycan; Polymeric immunoglobulin receptor (PIgR); Putative phospholipase B-like 1; Platelet factor 4 (PF4); Placenta growth factor (PlGF); Plasminogen; Serum paraoxonase/arylesterase 1 (PON 1); Serum paraoxonase/arylesterase 2 (PON 2); Serum paraoxonase/lactonase 3 (EC 3.1.1.2) (EC 3.1.1.81) (EC 3.1.8.1); Periostin (PN); Peptidyl-prolyl cis-trans isomerase B (PPIase B); Peroxiredoxin-4; Prolargin; Bone marrow proteoglycan (BMPG); Major prion protein (PrP); Prolactin (PRL); Vitamin K-dependent protein C; Properdin (Complement factor P); Presenilin-1 (PS-1); Protein patched homolog 1 (PTC1); Pleiotrophin (PTN); Receptor-type tyrosine-protein phosphatase C; Stromal cell-derived factor 1 gamma; Liver-specific organic anion transporter 3TM13 (Organic anion transporter LST-3c); SLCO1A2 protein; Mannan-binding protein (Fragment); 60S ribosomal protein L22; 60S ribosomal protein L29 (Cell surface heparin-binding protein HIP); Roundabout homolog 1 (H-Robo-1); R-spondin-1 (hRspo1); R-spondin-2 (hRspo2); R-spondin-3 (hRspo3); R-spondin-4 (hRspo4); 40S ribosomal protein SA; Solute carrier family 12 member 9; Sodium-dependent phosphate transporter 2; Solute carrier family 22 member 1 (hOCT1); Solute carrier family 22 member 7 (hOAT2); Solute carrier family 22 member 18; Sodium-coupled neutral amino acid transporter 3 (N-system amino acid transporter 1); Sodium-coupled neutral amino acid transporter 4; Zinc transporter ZIP4; Electrogenic sodium bicarbonate cotransporter 1 (kNBC1); Serum amyloid A protein (SAA); Serum amyloid P-component (SAP); Sodium channel protein type 5 subunit alpha (HH1); Stromal cell-derived factor 1 (SDF-1); Semaphorin-5A; Semaphorin-5B; Secreted frizzled-related protein 1 (FRP-1); Sonic hedgehog protein (SHH); Beta-galactoside alpha-2,6-sialyltransferase 1; Slit homolog 1 protein (Slit-1); Slit homolog 2 protein (Slit-2); Antileukoproteinase (ALP); Synaptogyrin-1; Superoxide dismutase; Extracellular superoxide dismutase; Sortilin; Sclerostin; Stabilin-2; Metalloreductase STEAP4; Stromal interaction molecule 1; Alpha-synuclein; Microtubule-associated protein tau; Teneurin-1 (Ten-1); Tenascin (TN); Tenascin-X (TN-X); Tissue factor pathway inhibitor (TFPI); Transferrin receptor protein 1 (TR); Transferrin receptor protein 2 (TfR2); Transforming growth factor beta receptor type 3; Transforming growth factor beta-1 (TGF-beta-1); Transforming growth factor beta-2 (TGF-beta-2); Protein-glutamine gamma-glutamyltransferase 2 (TGase-2); Thioredoxin (Trx); Prothrombin; Thyroglobulin (Tg); Metalloproteinase inhibitor 3; T-cell immunomodulatory protein (Protein TIP); Tumor necrosis factor ligand superfamily member 13; Tumor necrosis factor (TNF-alpha); Tissue-type plasminogen activator (t-PA); Tumor necrosis factor receptor superfamily member 11B; Serotransferrin (Transferrin); Lactotransferrin (Lactoferrin); Trypsin-1; Tryptase alpha/beta-1 (Tryptase-1); Tryptase beta-2 (Tryptase-2); Tumor necrosis factor-inducible gene 6 protein; Thrombospondin-1; Thrombospondin-2; Thrombospondin-3; Thrombospondin-4; Transthyretin (ATTR); Urokinase-type plasminogen activator (uPA); Vascular endothelial growth factor (VEGF); Vascular endothelial growth factor A (VEGF-A); Vascular endothelial growth factor B (VEGF-B); Vascular endothelial growth factor receptor 1 (VEGFR-1); Vascular endothelial growth factor receptor 2 (VEGFR-2); Vitamin D-binding protein (DBP); Vitronectin; von Willebrand factor (vWF); Proto-oncogene Wnt-1; Fractalkine (C-X3-C motif chemokine 1); Lymphotactin; Xanthine dehydrogenase/oxidase; Zinc transporter 1 (ZnT-1); and Protein Z-dependent protease inhibitor (PZI).

### Treatment and Prevention of Viral Infection

In some embodiments, there is provided a method of treating or preventing a viral infection in a subject in need thereof comprising administering to the subject an effective amount of one or more heparin or heparan sulfate compositions produced by methods described herein. In some embodiments, the heparin or heparan sulfate inhibits viral attachment to a cell. In some embodiments, the heparin or heaparan sulfate lacks anti-coagulant or anti-clotting activity. In some embodiments, the viral infection comprises a Adenoviridae such as, an Adenovirus; a Herpesviridae such as a Herpes simplex, type 1, a Herpes simplex, type 2, a Varicella-zoster virus, an Epstein-barr virus, a Human cytomegalovirus, a Human herpesvirus, type 8; a Papillomaviridae such as a Human papillomavirus; a Polyomaviridae such as a BK virus or a JC virus; a Poxviridae such as a Smallpox; a Hepadnaviridae such as a Hepatitis B virus; a Parvoviridae such as a Human bocavirus or a Parvovirus; a Astroviridae such as a Human astrovirus; a Caliciviridae such as a Norwalk virus; a Picomaviridae such as a coxsackievirus, a hepatitis A virus, a poliovirus, a rhinovirus; a Coronaviridae such as a Severe acute respiratory syndrome virus or a COVID-19 virus; a Flaviviridae such as a Hepatitis C virus, a yellow fever virus, a dengue virus, a West Nile virus; a Togaviridae such as a Rubella virus; a Hepeviridae such as a Hepatitis E virus; a Retroviridae such as a Human immunodeficiency virus (HIV); a Orthomyxoviridae such as an Influenza virus; a Arenaviridae such as a Guanarito virus, a Junin virus, a Lassa virus, a Machupo virus, a Sabia virus; a Bunyaviridae such as a Crimean-Congo hemorrhagic fever virus; a Filoviridae such as a Ebola virus, a Marburg virus; a Paramyxoviridae such as a Measles virus, a Mumps virus, a Parainfluenza virus, a Respiratory syncytial virus, a Human metapneumovirus, a Hendra virus, a Nipah virus; a Rhabdoviridae such as a Rabies virus; a Hepatitis D virus; or a Reoviridae such as a Rotavirus, a Orbivirus, a Coltivirus, a Banna virus infection. In some embodiments, treatment or prevention of the viral infection reduces one or more symptoms such as fever, diarrhea, fatigue, shortness of breath, or pain.

### Definitions

The term “glycosaminoglycan” or “GAG” as used herein refers to long unbranched polysaccharides consisting of a repeating disaccharide unit. The repeating unit (except for keratan) consists of an amino sugar (N-acetylglucosamine or N-acetylgalactosamine) along with a uronic sugar (glucuronic acid or iduronic acid) or galactose.

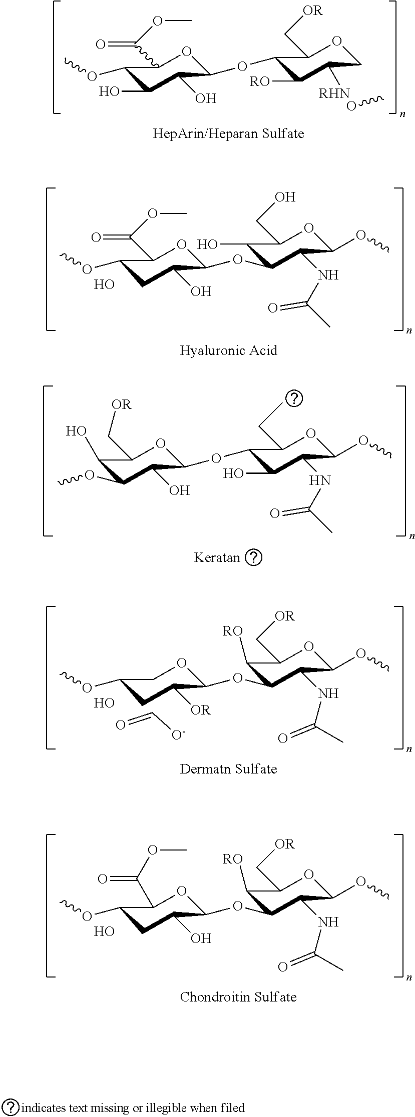

The term “proteoglycan” as used herein refers to proteins that are heavily glycosylated. The basic proteoglycan unit comprises a core protein with one or more covalently attached glycosaminoglycan or GAG chains.

The term “core protein” as used herein refers to a protein component of a proteoglycan.

The term “heparin” as used herein refers to a glycosaminoglycan made of repeating disaccharide units comprising one or more of β-D-glucuronic acid (GlcA), 2-deoxy-2-acetamido-α-D-glucopyranosyl (GlcNAc), α-L-iduronic acid (IdoA), 2-O-sulfo-α-L-iduronic acid (IdoA2S), 2-deoxy-2-sulfamido-α-D-glucopyranosyl (GlcNS), 2-deoxy-2-sulfamido-α-D-glucopyranosyl-6-O-sulfate (GlcNS6S) or 2-deoxy-2-sulfamido-α-D-glucopyranosyl-3,6-O-disulfate (GlcNS3S6S) or 2-deoxy-2-sulfamido-α-D-glucopyranosyl-3-O-sulfate (GlcNS3S).

The term “heparan sulfate” as used herein refers to a linear polysaccharide with the structure. Heparan sulfate is made of repeating disaccharide units. The repeating disaccharide units can comprise one or more of β-D-glucuronic acid (GlcA), 2-deoxy-2-acetamido-α-D-glucopyranosyl (GlcNAc), α-L-iduronic acid (IdoA), 2-O-sulfo-α-L-iduronic acid (IdoA2S), 2-deoxy-2-sulfamido-α-D-glucopyranosyl (GlcNS), 2-deoxy-2-sulfamido-α-D-glucopyranosyl-6-O-sulfate (GlcNS6S) or 2-deoxy-2-sulfamido-α-D-glucopyranosyl-3,6-O-disulfate (GlcNS3S6S) or 2-deoxy-2-sulfamido-α-D-glucopyranosyl-3-O-sulfate (GlcNS3S).

The term “chondroitin sulfate” as used herein refers to a linear polysaccharide with the structure. Chondroitin sulfate is made of repeating dissacharide units. The repeating disaccharide units can comprise one or more of N-acetylgalactosamine (GalNAc), N-acetylgalactosamine-4-sulfate (GalNAc4S), N-acetylgalactosamine-6-sulfate (GalNAc6S), N-acetylgalactosamine-4,6-disulfate (GalNAc4S6S) and β-D-glucuronic acid (GlcA), D-glucuronic acid-2-sulfate (GlcA2S), D-glucuronic acid-3-sulfate (GlcA3S), L-iduronic acid (IdoA), L-iduronic acid-2-sulfate (IdoA2S).

The terms “sulfation pattern”, “defined pattern of sulfation”, and “defined modification pattern” as used herein refer to enzymatic modifications made to the glycosaminoglycan including but not limited to include sulfation, deacetylation, and epimerization. This also includes heparin and heparan sulfate compositions having a defined disaccharide composition.

The term “genetically modified cell line” as used herein refers to a cell line with specific modifications made to the genome of the cell line. In some embodiments, the cell line is mammalian. In some embodiments, the cell line is human or murine. In some embodiments, the modifications comprise genetic knockouts, whereby the cell line becomes genetically deficient for one or more genes. In some embodiments, the modifications comprise making transgenic cell lines, whereby the cell line obtains genetic material not present in the wildtype cell line or genetic material under the control of active promoter.

The term “genetically deficient” as used herein refers to a genome that is modified to be missing one or more genes of interest. In some embodiments, the modification is made using a cre/lox system, CRISPR, siRNA, shRNA, antisense oligonucleotide, miRNA, or other genetic modification or mutagenesis method known in the art.

The term “transgenic” as used herein refers to a genome that is modified to include additional genetic material encoding one or more genes of interest. In some embodiments, the modification is made using transfection, infection with a virus, cre/lox knock-in, CRISPR/cas mediated knock-in, or other method of introducing genetic material to a cell that is known in the art.

The terms “subject”, “individual”, “recipient”, “host”, and “patient”, are used interchangeably herein and refer to any mammalian subject for whom diagnosis, treatment or therapy is desired, particularly humans. “Mammal” for purposes of treatment refers to any animal classified as a mammal, including humans, domestic and farm animals, and laboratory, zoo, spots, or pet animals, such as dogs, horses, cats, cows, sheep, goats, pigs, mice rats, rabbits, guinea pigs, monkeys, etc. In some embodiments, the mammal is human.

As used herein, the terms “treatment”, “treating” and the like, refer to administering an agent or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment”, as used herein, may include treatment of a disease in a mammal, particularly in a human and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of a disease, including any objective or subjective parameter such as abatement; remission; diminishing of symptoms or making the disease more tolerable to the patient; slowing in the rate of degeneration or decline; or making the final point of degeneration with less debilitation. The treatment or amelioration of symptoms can be based on objective or subjective parameters; including the results of an examination by a physician. Accordingly, the term “treating” includes the administration of the compounds or agents disclosed hereinto prevent or delay, to alleviate, or to arrest or inhibit development of the symptoms of conditions associated with the disease. The term “therapeutic effect refers to the reduction, elimination, or prevention of the disease, symptoms of the disease, or side effects of the disease in the subject.

“In combination with”, “combination therapy” and “combination products” refer, in certain embodiments, to concurrent administration to a patient of a first therapeutic and the compounds used herein. When administered in combination, each component can be administered at the same time or sequentially in any order at different points in time. Thus, each component can be administered separately but sufficiently closely in time as to provide the desired therapeutic effect.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or in the case of an aerosol composition, gaseous.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means that the amount that, when administered to a subject for treating a disease, is sufficient to effect treatment for that disease.

The term “substantially free” as used herein means most or all of one or more of a contaminant, such as the materials with which it typically associates with in nature, is absent from the composition. Thus a heparin or heparan sulfate composition with defined modification patterns described herein that is “substantially free” from one or more contaminating glycosaminoglycans that do not have the desired defined modification pattern and/or biological and/or therapeutic effect has no or little of the contaminant. For example, a heparan sulfate composition is “substantially free” from a contaminant such as other glycosaminoglycans such as: chondroitin sulfate, keratan sulfate and/or hyaluronic acid; nucleic acids; and/or proteins, found with the heparan sulfate composition in nature, has very little or none of the contaminant, for example less than 5%, less than 4%, less than 3%, less than 2%, less than 1%, or less than 0.5% of the composition is made up by the contaminant. In some embodiments, the composition is 60%, 70%, 80%, 85%, 90%, 95%, 96%, 97%, 98%, 99%, or 100% free from one or more of a contaminating glycosaminoglycan, nucleic acids, and or proteins. In some embodiments, the composition is at least 95% free from contaminating glycosaminoglycans, nucleic acids, and or proteins. In some embodiments, the composition is at least 99% free from contaminating glycosaminoglycans, nucleic acids, and or proteins.

The term “substantially pure” as used herein means that the composition is free of most or all of the materials with which it typically associates with in nature. Thus a “substantially pure” glycosaminoglycan and/or heparan sulfate composition with defined modification patterns described herein does not include other contaminating glycosaminoglycan and/or heparan sulfate compositions that do not have the desired defined modification pattern and/or biological and/or therapeutic effect. For example, a “substantially pure” heparan sulfate composition is free from most other glycosaminoglycans such as: chondroitin sulfate, keratan sulfate and/or hyaluronic acid; nucleic acids; and/or proteins, found with the heparan sulfate composition in nature. In some embodiments, the composition is 60%, 70%, 80%, 85%, 90%, 95%, 96%, 97%, 98%, 99%, or 100% free from contaminating glycosaminoglycans, chondroitin sulfate, dermatan sulfate, keratan sulfate, nucleic acids, and or proteins. In some embodiments, the composition is 95% free from contaminating glycosaminoglycans, chondroitin sulfate, dermatan sulfate, keratan sulfate, nucleic acids, and or proteins. In some embodiments, the composition is 99% free from contaminating glycosaminoglycans, chondroitin sulfate, dermatan sulfate, keratan sulfate, nucleic acids, and or proteins. In some embodiments, the composition is greater than 99% free from contaminating glycosaminoglycans, chondroitin sulfate, dermatan sulfate, keratan sulfate, nucleic acids, and or proteins.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

### Example 1: Methods

Cell Culture. Cell lines derived from the Furth murine mastocytoma were grown in DME/F12 medium (1:1) (Cytiva) supplemented with 15 percent fetal bovine serum (FBS, BioWest). CHO-S cells (ThermoFisher) were grown in CD CHO medium (Gibco) with 8 mM GlutaMAX (Gibco). 293F cells (ThermoFisher) were grown in FreeStyle 293 medium (Gibco). HeLa-S3 cells (Sigma) were grown in EX-CELL HeLa medium (Sigma) with 6 mM GlutaMAX. Engineered MST cells were later grown in CD CHO, CDM4NS0 and SFM4MAb serum free medium (Cytiva). Cells were typically grown in 30 mL of culture medium in shaker flasks (125 rpm) at 37° C., under a 5% CO2 in 95% air. Viable cell density was determined on each day using a hemocytometer and trypan blue to determine the integrated viable cell density.

Gene Expression Determination. Primary mouse mast cells were generated by extracting bone marrow cells from the femurs of 5-8-wk-old mice and culturing cells in RPMI 1640 medium (Invitrogen) supplemented with 10% inactivated FBS (Thermo Fisher Scientific), 25 mM HEPES (pH 7.4), 4 mM L-glutamine, 0.1 mM nonessential amino acids, 1 mM sodium pyruvate, 50 uM 2-ME, 100 IU/mL penicillin, and 100 ug/mL streptomycin. Recombinant murine IL-3 (1 ng/mL; R&D Systems) and recombinant murine stem cell factor (SCF; 20 ng/mL; R&D Systems), both shown to support the in vitro growth and differentiation of the mast cell precursor, were also included. Mouse mast cells derived from bone marrow cells with SCF and IL-3 become mature after 4 wk of culture in vitro. After 4 wk, mast cells were consistently generated as confirmed by the expression of CD117 (c-Kit) and FcεRI by flow cytometry; cell maturation was confirmed by metachromatic staining with toluidine blue. The purity of MCs was greater than 98%.

The disaccharide composition of HS purified from bone marrow derived cells at 0 to 4 weeks into the differentiation protocol was determined by liquid chromatography/mass spectrometry. RNA was prepared from 0.5×106 undifferentiated and week 3 differentiated cells using the RNeasy kit (Qiagen) with RNase-Free DNase (Qiagen). MST cells grown in DME/F12 with 15% FBS were prepared in parallel for comparison. RNA sample quality was assessed by TapeStation (Agilent) with RNA Integrity Number greater than 8.0 deemed acceptable for generating sequencing libraries. The TruSeq Stranded mRNA Sample Prep Kit (Illumina) was used to generate sequencing libraries. RNA libraries were multiplexed and sequenced with 100 base pair paired end reads to a depth of approximately 50 million reads per sample on an Illumina HiSeq4000.

Heparan Sulfate Production. Cells were grown in suspension in shaker flasks. Cultures were typically seeded at 0.2×10⁶ cells/mL in 30 mL of culture medium in shaker flasks (125 mL) at 37° C. under an atmosphere of 5% CO2/95% air for seven days. For larger preparations, cells were seeded in 100-300 mL of medium in multiple 1 L flasks. Cells and medium were separated by centrifugation (9000×rpm, 10 min). The cell pellet was resuspended in buffer (25 mM sodium acetate, pH 6.0, 0.25 M NaCl) plus 0.1% Triton X100 (wt/vol) and 0.5 mg/mL Pronase (Sigma) and incubated with shaking overnight at 37° C. Conditioned medium and protease digested cells were mixed, filtered through a 0.45 μm PES filter and applied to 0.5 mL DEAE-Sephacel columns, equilibrated and washed with DEAE wash buffer (25 mM sodium acetate, pH 6.0, 0.25 M NaCl) and eluted with the same buffer containing 2 M NaCl. The eluate was desalted on PD10 columns (Cytiva) for small volumes or by dialysis for large volumes and then dried by lyophilization. The dried product was reconstituted in water, then digested with micrococcal nuclease (Worthington) overnight at 37° C. followed by Pronase digestion for 3 hours at 37° C. Beta-elimination was performed to liberate the HS/heparin from proteoglycans by addition of NaOH to 0.4 M with overnight incubation at 4° C. After the sample was neutralized with acetic acid, it was diluted with water and reapplied to DEAE-Sephacel, then washed and eluted as described above. The eluate was desalted and lyophilized. Protein and DNA content were measured with the BCA protein assay and by UV absorbance. The nuclease and Pronase digests were repeated as needed to eliminate residual nucleic acid or protein contamination.

Heparan Sulfate Characterization. HS was quantified using the carbazole assay. Briefly, 100 μL of sample was mixed with 10 μL of 4 M ammonium sulfamate and 0.5 mL of 25 mM sodium tetraborate in sulfuric acid. The samples were heated to 95° C. for 5 min, cooled to room temperature, and 20 μL of 0.1% carbazole in 95% ethanol was added. The samples were heated to 95° C. for 15 min. Absorbance at 520 nm was then measured. Glucuronic acid was used to construct a standard curve. The molecular weight of the sodium salt of HS was calculated based on the glucuronic acid content and the average disaccharide molecular weight of the preparation as determined by disaccharide composition analysis.

HS disaccharide composition was determined by digestion of the polysaccharide and quantification of the resulting disaccharides by UPLC. Briefly, 1 μg of HS was diluted to 120 μL in 40 mM ammonium acetate and 3.3 mM calcium acetate, pH 7.0 and treated with 2 mU each of heparin lyases I, II and III overnight at 37° C. The samples were then dried in a SpeedVac and reconstituted in 10 μL of 23.5 mM 2-aminoacridone in 85% DMSO/15% acetic acid and incubated at 37° C. for 10 min. NaBH3CN (10 μL of 1 M in 85% DMSO/15% acetic acid) was added and the sample was incubated at 37° C. overnight. The reaction was terminated by addition of 180 μL of 50 mM acetic acid. Samples were loaded onto an Acquity UPLC (Waters) equipped with a fluorescence detector and a HSS C18 column (1.8 μm beads, 2.1×100-mm inner diameter) equilibrated in 0.1 M ammonium acetate. Disaccharides were resolved with a methanol gradient and were quantified relative to disaccharide standards (Iduron). In some cases, composition was determined by tagging the disaccharides with aniline instead of 2-aminoacridone and the disaccharides were quantified by LC/MS using isotopically labeled disaccharide standards for quantification. For detection of CS, glycosaminoglycans were purified from cell culture as described above for HS and purified samples were digested with chondroitinase ABC (Amsbio), labeled with aniline and analyzed by LC/MS using CS standards.

The anticoagulant activity of HS was determined using an anti-Xa assay. Human antithrombin (Enzyme Research Laboratories) was diluted to 66 μg/mL, Factor Xa (Enzyme Research Laboratories) was diluted to 0.4 μg/mL and substrate S-2765 (Chromogenix) was diluted to 0.5 mg/mL in assay buffer (25 mM HEPES, pH 7.4, 150 mM NaCl, 0.10% BSA). For each sample, 5 ng of HS was diluted to 96 L in assay buffer, then 24 μL of antithrombin was added and then 50 μL of the sample was pipetted into duplicate wells of a 96-well plate. Factor Xa solution was added (50 μL) to each well followed by 50 μL of the substrate S-2765. The hydrolysis of the substrate was measured at OD450 and the rate of reaction was determined from the slope. A standard curve of pharmaceutical unfractionated heparin (APP Pharmaceuticals) was used to determine the anti-Xa activity of the samples. Anti-Xa yield was determined by multiplying the specific anti-Xa activity and the HS yield of the culture.

Genetic Engineering. Gene targeting was accomplished using CRISPR/Cas9 with sgRNAs designed using Synthego's CRISPR Design Tool and sgRNAs and Cas9 from Synthego. sgRNAs were designed in pairs to excise large segments (>200 bp) of the gene to accelerate the identification of useful clones by PCR. Viral transductions were performed using lentivirus with ORF and lentiviral constructs produced by GenScript. Constructs for transfection were produced by GenScript.

Transductions and transfections were performed on cells grown in DME/F12 containing 15% FBS. Transductions were performed on MST cells by spinoculation with 10⁵ cells in 0.5 mL of growth medium plus 8 μg/mL polybrene and viral particles at MOI 100. Tubes were spun at 800×g for 20 min at room temperature, then the cells were transferred into fresh medium and cultured overnight. The cells were transferred to fresh medium again after 24 hr. Transfections were performed on a Neon electroporation device (ThermoFisher) with 10⁶ cells and 5 μg of plasmid construct in 100 μL of Buffer R. MST cells were electroporated at 1700 U for 20 msec (1 pulse). For CRISPR mediated knockout, the cells were transfected with 180 pmol each of paired sgRNAs and 20 pmol of Cas9 cDNA.

Verification of transgene integration was performed by PCR on genomic DNA with primers specific to the sequence flanking the targeted region of interest. Successful gene mutations by CRISPR were verified by PCR on genomic DNA using primers that flank the mutation site. Successful excision of a block of DNA resulted in a shift of the PCR product to a lower size on the gel. Genotyping was performed in this way for both mixed populations and single cell-derived colonies.

Screening Colonies by Heparan Sulfate Characterization. An abbreviated HS purification strategy was used to streamline screening of colonies for anti-Xa activity. Single cell colonies were grown in a 6-well dish containing 2 mL of DME/F12 containing 15% FBS for five days. In some cases, 100 μL of cell suspension was collected, washed in PBS, and used for a BCA protein assay. The remaining content of the well was adjusted to 0.1% Triton X100 and 0.5 mg/mL Pronase and digested overnight at 37° C. The digested product was passed over a bed of 100 μL DEAE-Sephacel, washed with DEAE wash buffer and eluted with 0.5 mL DEAE elution buffer. The eluate was used directly in the anti-Xa activity assay with an equal volume of DEAE elution buffer added to the standards. Activity was expressed on a per well or a per cellular protein basis. After colonies of interest were identified using this method, the colonies were grown up in shaker flasks and the HS was prepared and characterized as described above.

Anticoagulation assays. The anticoagulant effect of HS was assessed in vivo by injection of C57BL/6J male mice (8 weeks of age) with PBS, 3 mg/kg UFH (APP) or 3 mg/kg of B 7A HS (n=4 per group) via subcutaneous injection. Blood was collected from the tail incisions into tubes with sodium citrate buffer at 30, 60 and 180 min after injection. The cells were removed by centrifugation and the supernatant was assayed for anti-Xa activity as described above.

### Example 2: Engineering MST Cell LInes

All mammalian cells produce HS although the degree of sulfation and relative content of uronic acid epimers differs by cell type. The degree of sulfation and iduronic acid content of HS is typically much less than that found in heparin. For example, HS in CHO cells contains 33% GlcNS, 5% 6S, 14% 2S, and lacks 3-O-sulfation entirely. The iduronic acid content is also very low. In contrast, heparin contains 83% GlcNS, 83% 6S, 69% 2S (FIG. 1B). Some types of cells have higher sulfate and iduronic acid content (e.g. macrophages), but HS from mammalian cells commonly used for bioproduction (CHO-S, HeLa-S3, 293F) is much less modified than porcine unfractionated heparin. In contrast, HS produced by a mouse mastocytoma (MST) cell line has sulfate content and iduronic acid content comparable to heparin (FIG. 1B). However, MST HS lacks 3-O-sulfation and anticoagulant activity. MST cells grow in suspension and have been previously engineered by introduction of HS3ST1 in partially successful attempts to make recombinant heparin.

Mammalian cells also produce CS. The initial steps of heparin/HS and CS biosynthesis involve the assembly of a linkage tetrasaccharide (GlcA-Gal-Gal-Xyl-) attached to serine residues in core proteins. Thus, CS production may compete with heparin/HS for precursor metabolites, such as nucleotide sugars and PAPS, the high energy sulfate donor for all sulfation reactions. Previous studies found that MST cells produced significant quantities of CS that was both stored intracellularly and secreted into the media. CS co-purifies with HS and must be removed by exhaustive digestion with chondroitinase ABC. To streamline purification and eliminate the possibility of contamination, CS biosynthesis was genetically eliminated from the cell lines. To quantitate the relative production of HS and CS, samples were treated with heparin lyases or chondroitinases ABC and the resulting disaccharides were quantitated by LC/MS (FIG. 1C)

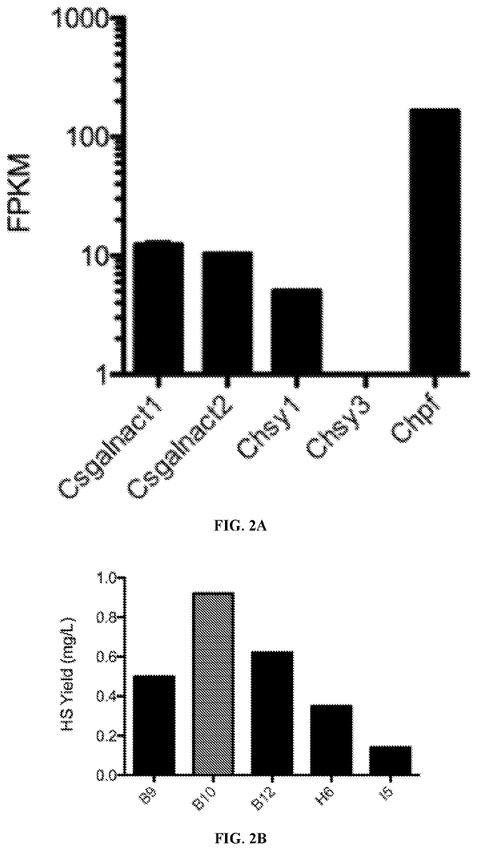

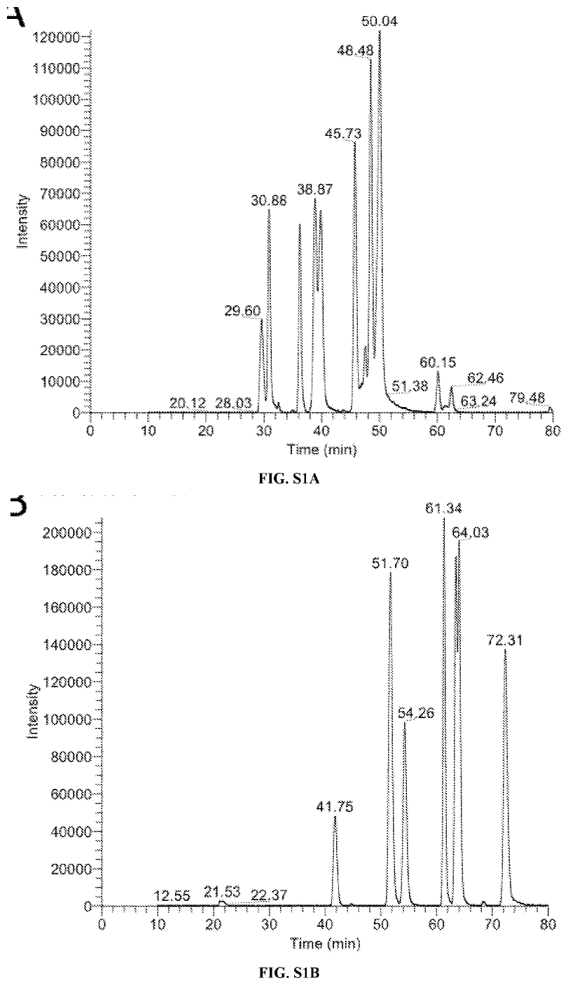

RNAseq transcript analysis revealed that MST cells express CS biosynthetic enzymes chondroitin sulfate N-acetylgalactosaminyltransferase 1 and 2 (Csgalnact1 and Csgalnact 2), chondroitin sulfate synthase 1 (Chsy1), and chondroitin polymerizing factor (Chpf) (FIG. 2A).

Inactivation of Chpf using CRISPR/Cas gene targeting did not eliminate CS synthesis in CHO-S cells. Thus, CRISPR/Cas9 was used to simultaneously target Csgalnact1, Csgalnact2 and Chsy1 creating a mixed population called MST17. Screening by PCR of 160 clones identified five clones that had all three genes inactivated. Clone B10 lacked any CS (FIG. S1) and had the highest level of HS production (FIG. 2B).

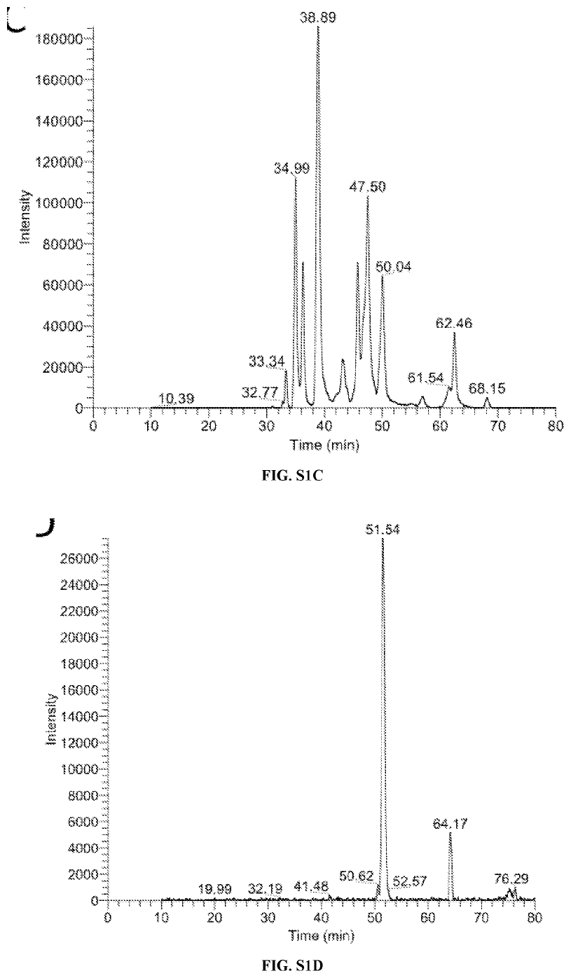

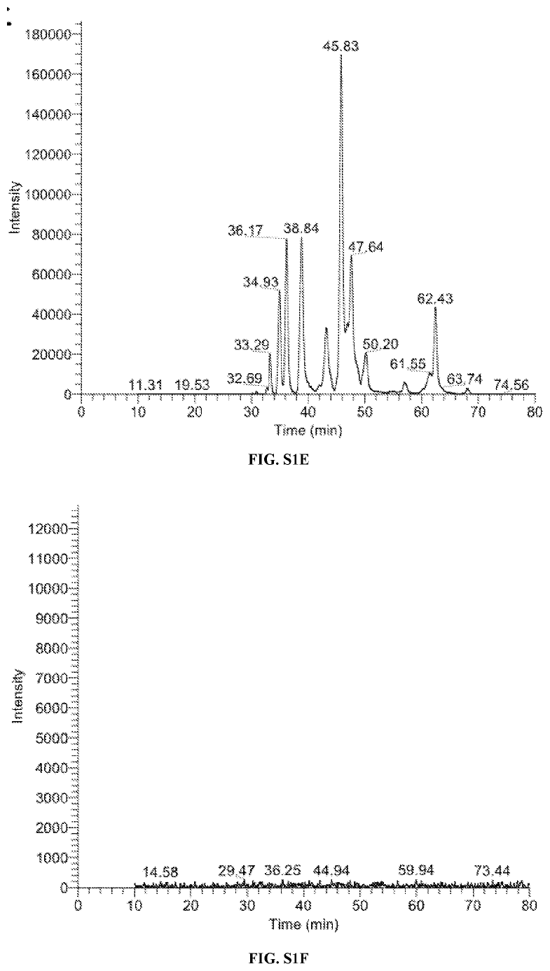

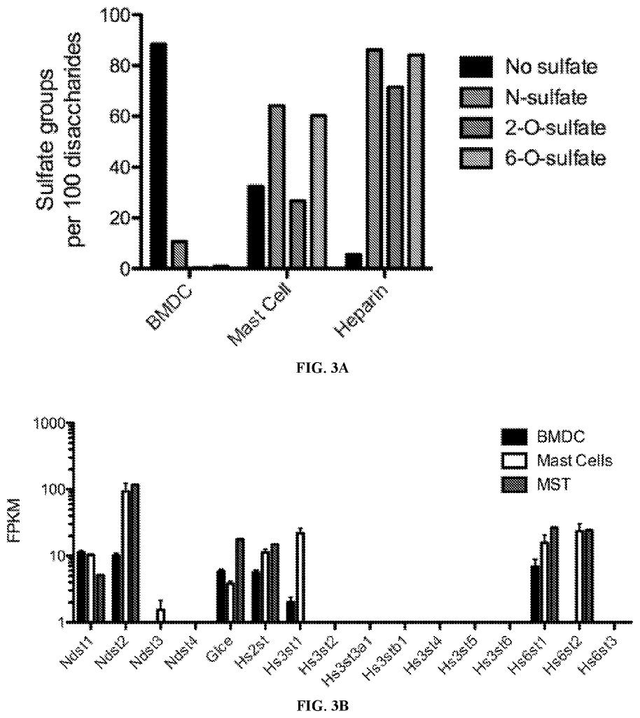

Previous studies have reported the lack of 3-O-sulfate in MST heparin and shown that overexpression of Hs3st1 increased the anti-Xa activity of fractionated MST HS from 20 U/mg to 50-60 U/mg, however, the US Pharmacopeia requirement for anti-Xa activity for pharmaceutical heparin is not less than 180 U/mg. Assuming that there may be other enzyme deficiencies, comparative gene expression analyses were employed to compare RNA expression in MST cells and bone marrow derived cells before and after differentiation into mast cells. Pharmaceutical heparin is derived from porcine intestinal mucosa but porcine-derived mast cells were difficult to procure and data regarding porcine mast cell gene expression unavailable. Transcript levels in murine derived mast cells could be compared directly with transcript levels in MST cells so mouse mast cells were analyzed. Mouse bone marrow derived cells (BMDC) produced HS largely devoid of sulfate while HS from differentiated mast cells had higher sulfate content albeit only 64% of the sulfate content in pharmaceutical heparin (FIG. 3A). Fractionation of crude porcine-derived heparin to enrich for anticoagulant activity likely accounts for the higher sulfate content in pharmaceutical heparin compared to the heparin purified from differentiated mast cells.

RNAseq analyses verified the lack of Hs3st1 expression in MST cells and showed that Hs3st1 was expressed in differentiated mast cells more than an order of magnitude higher than BMDC (FIG. 3B). These analyses also confirmed that the major heparin/HS polymerization and modification enzymes were present at significant levels in the MST cells relative to heparin producing mast cells including Ext1, Ext2, Ndst1, Ndst2, Glce, Hs2st, Hs6st1 and Hs6st2 (FIG. 3B). Other enzymes involved in metabolism and transport of activated sugar precursors (FIG. S2A) and HS core proteins were also expressed (FIG. S2B).

Since Hs3st1 overexpression was previously insufficient to produce high anti-Xa activity in MST cells and none of the other enzymes appeared to be lacking, it was speculated that artificially high enzyme expression levels may be required in tissue culture to achieve heparin-like sulfate content and anticoagulant activity. Previous analyses, showing HS purified from Hs3st1-transduced MST cells, suggest that overexpression of Hs3st1 produced sufficient 3-O-sulfation but may have lacked critical 6-0-sulfated residues. The above disaccharide composition analyses also indicated that 6-O-sulfate was lacking MST cells compared to unfractionated heparin (FIG. 1B). 2-O-sulfate is also reduced in MST cells compared to unfractionated heparin, but it appears that 2-O-sulfate is not required for heparin's high anticoagulant activity. The comparative expression analyses showed that Hs6st2 expression increases over 200-fold upon differentiation of BMDC to heparin-producing mast cells suggesting an important role for Hs6st2 in mast cell heparin production, however MST cells already express levels of Hs6st1 and Hs6st2 similar to differentiated mast cells, so there may not be a bias for either of these enzymes in MST cells. Likewise, Ndst2 expression increased ten-fold from BMDC to differentiated mast cells.

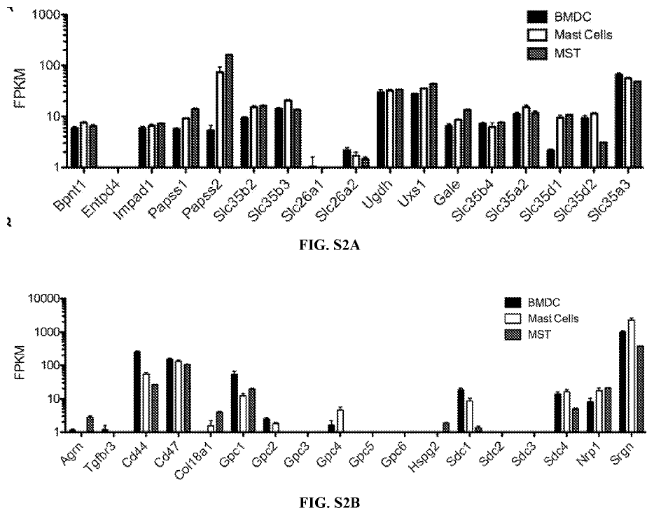

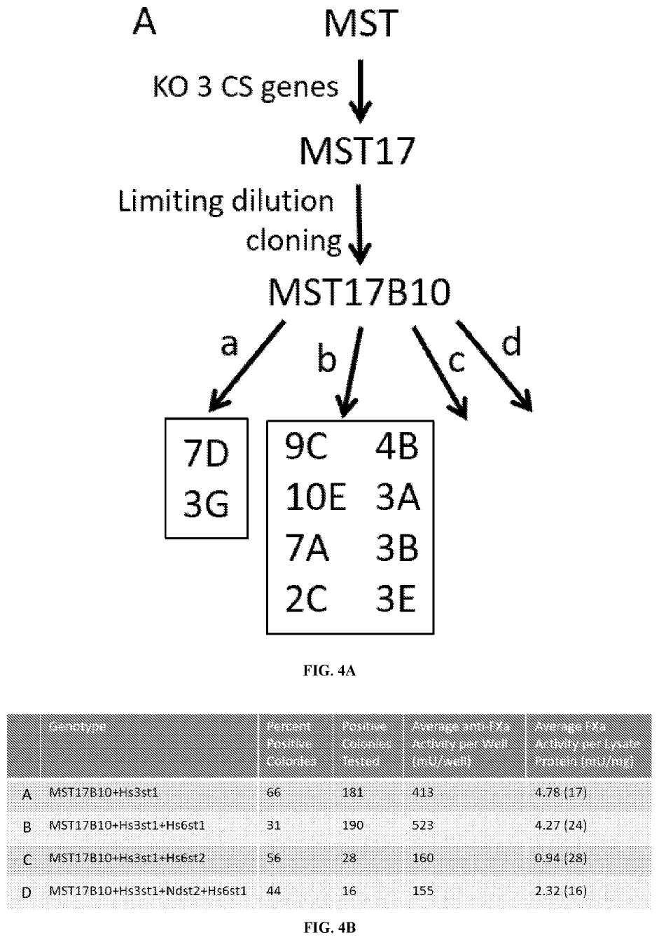

Based on these analyses, MST17B10 cells were transduced to overexpress Hs3st1 alone and in combination with Hs6st1, Hs6st2 and Ndst2 (FIG. 4A). Single cell colonies were isolated from the transduced populations by limiting dilution cloning and the genotypes were determined by PCR. HS was purified from these colonies growing in 6-well dishes and was assayed for anti-Xa activity. FIG. 4B shows average anti-Xa activity per well for single cell colonies from each combination of transduced genes. At first, total cellular protein was also measured for colonies to compensate for differences in cell number, however, the normalized values closely correlated with total activity in the wells, so total cellular protein was not measured in subsequent screening. In initial screening, the highest anti-Xa activities were observed in colonies transduced with Hs3st1 or Hs3st1 and Hs6st1 so additional colonies in those two groups were screened. The high anti-Xa activity in the colonies transduced exclusively with Hs3st1 highlights the importance of 3-O-sulfate and suggests that transduced Hs3st1 gene dosage may be a determining factor. Since high anti-Xa activity was observed in cells transduced with Hs3st1 with and without co-transduction of Hs6st1, there may be more than one way to achieve the configuration of 3-O- and 6-O-sulfated residues required to form the necessary frequency of antithrombin binding sites.

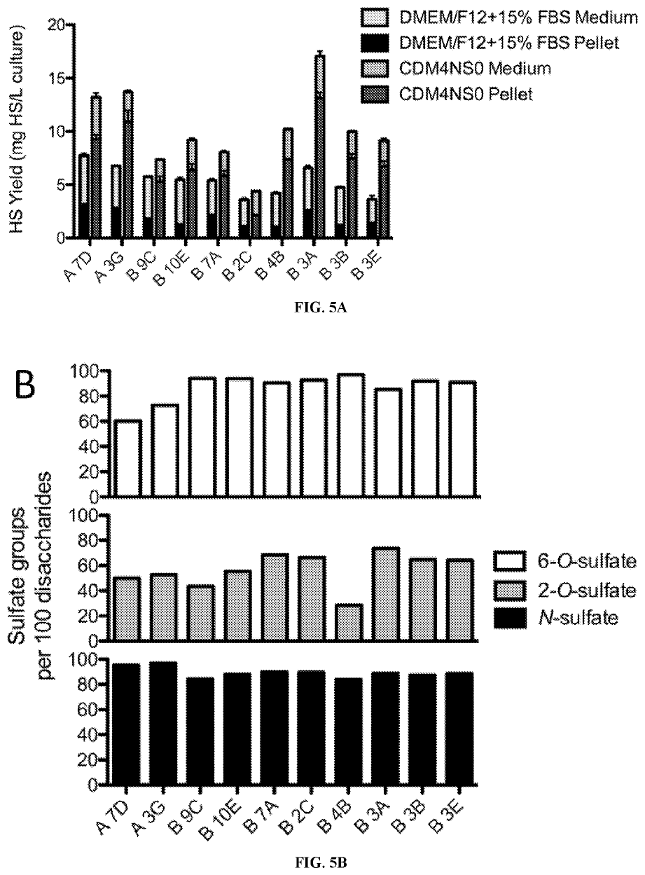

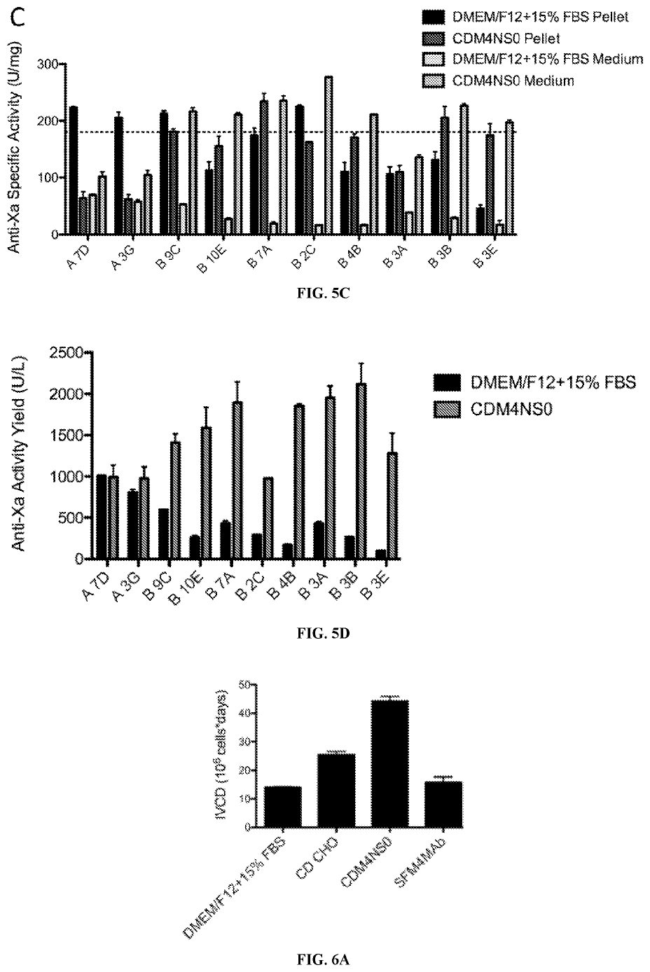

The ten best colonies with the highest total anti-Xa activity/well were expanded into shaker flask cultures in DME/F12+15% FBS for more extensive analysis. HS was purified and characterized from both the cell pellet and conditioned medium in these cultures. HS yield (FIG. 5A), sulfate content (FIG. 5B) and anti-Xa specific activity (FIG. 5C) varied by cell line. Importantly, cellular HS purified from the cell pellets exhibited high anti-Xa specific activity in some cases exceeding 200 U/mg. Anti-Xa specific activity was considerably lower in HS purified from the cell culture medium, however, the yields of HS were typically higher from the medium than from the pellets (FIG. 5A).

### Example 3: Transition Cells to Serum Free Media

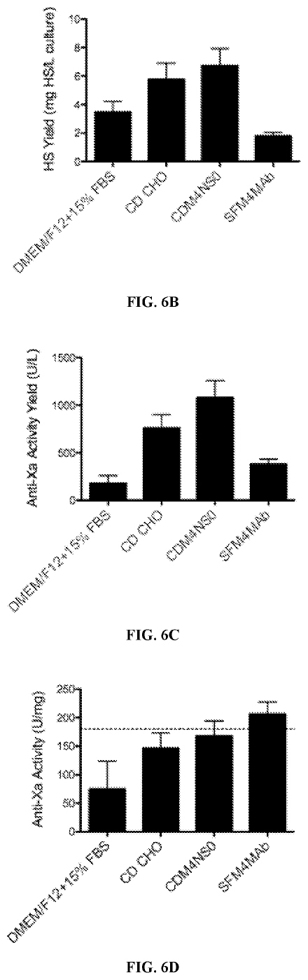

GMP production for use in humans requires that the cells be grown in serum free medium. B 3E cells were transitioned to three commercial serum free media and tested for cell growth (FIG. 6A), HS production (FIG. 6B) and anti-Xa activity yield (FIG. 6C) and specific activity (FIG. 6D). CDM4NS0 proved superior in all categories and so was chosen for further testing.

Growth of the candidate cell lines in CDM4NS0 resulted in a number of changes in HS production. In CDM4NS0, the yield of recombinant HS purified from the cell pellets increased compared to cells grown in DME/F12+15% FBS (FIG. 5A). The recombinant HS yields from the medium remained constant or decreased in excess of 50% among the different clones (FIG. 5A), however, the anti-Xa specific activity of HS purified from the medium increased in all clones and in some quite significantly (FIG. 5C). On the other hand, whereas anti-Xa specific activity in HS purified from the cell pellets remained constant or increased slightly in clones transduced with both Hs3st1 and Hs6st1, the anti-Xa specific activity of HS purified from the cell pellets of cells transduced with only Hs3st1 decreased (FIG. 5C). Consistent with this finding, composition analyses showed lower levels of 6-O-sulfation in the cell lines transduced with only Hs3st1 compared to cell lines transduced with Hs3st1 and Hs6st1 (FIG. 5B). Reduced anti-Xa activity may reflect a reduction of 6-O-sulfate groups, which are critical for activation of antithrombin, when the cells are grown in CDM4NS0.

Ultimately, the total cellular recombinant heparin will be purified including polysaccharides from the cell pellets and medium to produce the maximum yield of anti-Xa units. FIG. 5D shows that the total yield of anti-Xa units was significantly increased in CDM4NS0 for the clones transduced with Hs3st1 and Hs6st1 but was essentially unchanged for cell lines transduced with only Hs3st1. Perhaps more importantly, 6 of 8 clones transduced with both Hs3st1 and Hs6st1 produced HS with anti-Xa specific activity equal to or above 180 U/mg (FIG. 5C).

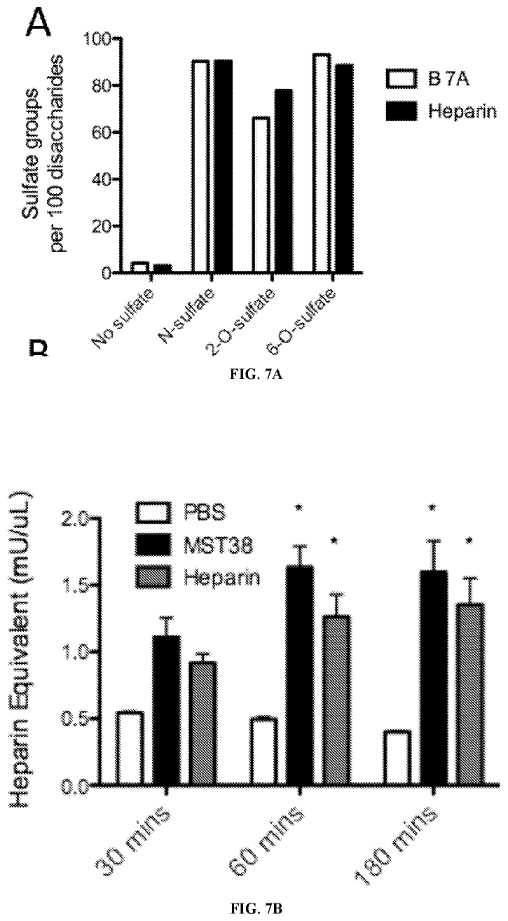

Clone B 7A was used to more fully characterize the recombinant HS produced in serum free medium. Clone B 7A was chosen because of the high anti-Xa specific activity in recombinant HS from both the cell pellet and the medium (FIG. 5C) and the relatively high overall yield of anti-Xa activity (FIG. 5D) in CDM4NS0. Clone B 7A was expanded in CDM4NS0 to multiple 1 L flasks and was allowed to grow for seven days before harvesting recombinant HS from the cell pellets and the media separately. Recombinant HS yield from the cell pellet and the conditioned medium was 6.0 and 0.72 mg/L of culture volume, respectively. The recombinant HS purified from the cell pellets had sulfate content similar to unfractionated heparin (FIG. 7A). Recombinant heparin purified from the cell pellets was also assayed for potency and molecular weight using USP reference standards. When titrated against antithrombin, B 7A recombinant HS showed 64.8 percent high affinity material compared to 42.6 percent for the USP heparin standard indicating that the cell engineering favored formation of antithrombin binding sites. B 7A HS performed similarly to unfractionated heparin in vivo. Mice (n=4) were administered a subcutaneous injection of B 7A HS, unfractionated heparin or an equal volume of saline. Blood was collected into citrated tubes at 30, 60 and 180 minutes after injection and anti-Xa activity was determined in the plasma from each sample (FIG. 7B). B 7A HS conferred greater anticoagulation to mouse plasma than porcine derived heparin as shown by the anti-Xa activity assay.

### Example 4: Heparin with Reduced Side Effects

Heparin induced thrombocytopenia (H.I.T.) is a serious side effect that can be fatal. H.I.T. is caused by a complex formed between heparin and platelet factor 4 (PF4). Reducing the PF4 binding affinity of heparin would reduce the risk of H.I.T. Therefore, cells were engineered to produce heparan sulfate with reduced binding affinity for PF4. Two strategies were used to reduce PF4 binding affinity. The first strategy was to alter the composition by reducing 2-sulfation. The second strategy was to alter the composition by reducing the frequency of certain 6-sulfated residues by overexpressing the SULF2 gene.

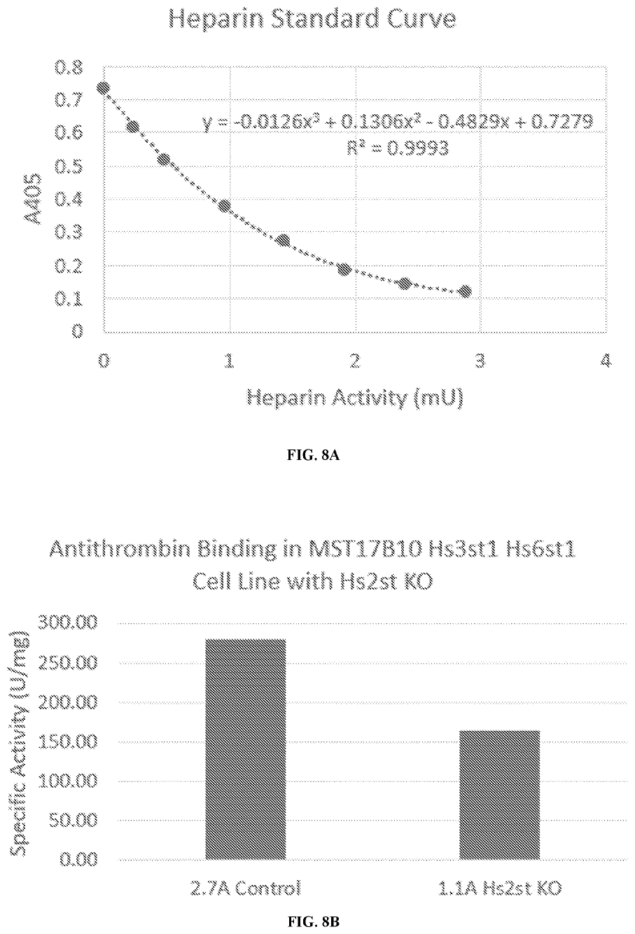

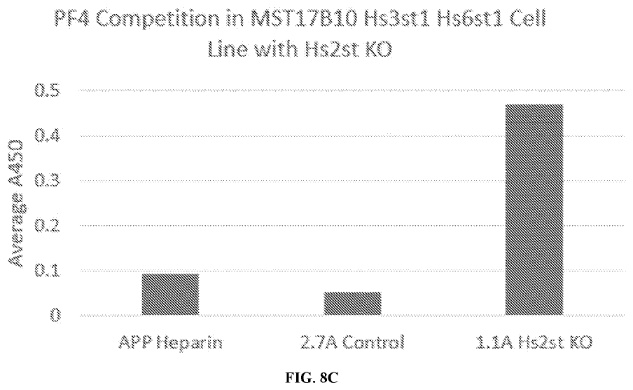

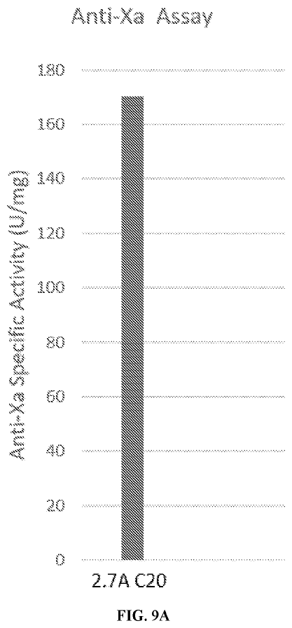

Reduced 2-sulfation: The 2.7A cell line was previously engineered to over express sulfotransferase enzymes Hs3st-1 and Hs62t-1 for producing heparan sulfate with increased anticoagulant activity. To reduce the platelet factor 4 (PF4) binding affinity of the heparan sulfate, 2-sulfation was decreased by genetically knocking out the HS2ST gene using CRSPR-Cas technology. Single clones were identified with reduced PF4 binding affinity, but which maintained high anticoagulant activity. FIGS. 8A-C show clone 1.1A. FIG. 8A is the standard curve for the Factor Xa activity assay (anticoagulant activity). FIG. 8B shows that anticoagulant activity was reduced relative to the parent cell line (2.7A) but was still over 150 U/mg. FIG. 8C shows the results of a PF4 competition binding assay. In the competition assay, PF4 binding to heparin immobilized on assay plates is determined in the presence of a competitor. FIG. 8C shows that PF4 binding to clone 1.1A was dramatically reduced compared to the parent cell line (2.7A) and pharmaceutical heparin (APP heparin) because binding to the immobilized heparin was significantly greater (and not reduced by competition).

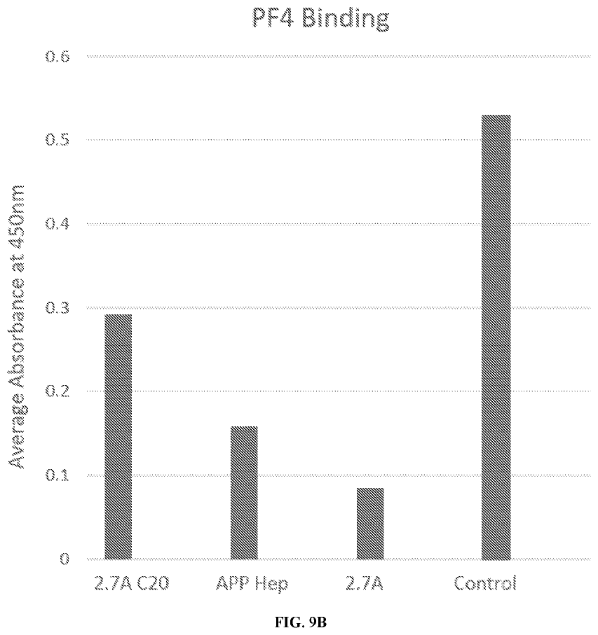

Sulf-2 overexpression: The 2.7A cell line was previously engineered to overexpress sulfotransferase enzymes Hs3st-1 and Hs62t-1 for producing heparan sulfate with increased anticoagulant activity. Extracellular Sulf-2 is an enzyme that reduces the frequency of 6-sulfated residues on heparin and heparan sulfate such that Factor Xa activity remains high. PF4 Binding affinity also depends on 6-sulfation so the SULF2 gene was overexpressed in 2.7A cells to reduce PF4 binding. FIG. 9A shows that cell line 2.7A-C20 (that overexpresses the SULF2 gene) maintains high antifactor Xa activity. FIG. 9B shows the results of a PF4 competition binding assay. In the competition assay, PF4 binding to heparin immobilized on assay plates is determined in the presence of a competitor. FIG. 9B shows that PF4 binding to clone C20 is reduced compared to the parent cell line (2.7A) and pharmaceutical heparin (APP heparin) because binding to the immobilized heparin was significantly greater (and not reduced by competition). The control is PF4 binding in the absence of any competitor.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of producing a heparin or heparan sulfate, the method comprising culturing a genetically modified mastocytoma cell line; and isolating the heparin or heparan sulfate from the cell line,
wherein the genetically modified cell line overexpresses one or more of heparan sulfate-glucosamine 3-sulfotransferae 1 (Hs3st1), heparan sulfate 6-O-sulfotransferase 1 (Hs6st1), heparan sulfate 6-O-sulfotransferase 2 (Hs6st2), and N-deacetylase N-sulfotransferase 2 (Ndst2), and Sulf-2.

2. The method of claim 1, wherein the genetically modified cell line is deficient for one or more of chondroitin sulfate N-acetylgalactosaminyltransferase 1 (Csgalnact1), chondroitin sulfate N-acetylgalactosaminyltransferase 2 (Csgalnact2), chondroitin sulfate synthase 1 (Chsy1), and Heparan sulphate 2-O-sulfotransferase (Hs2st).

3. The method of claim 1 or claim 2, wherein the mastocytoma cell line is selected from the group consisting of MST cells, P815 cells, MC/9 cells, SI/SI4 cells, 10P2 cells, 11P0-1 cells, and 10P12 cells.

4. The method of any one of claims 1 to 3, wherein the genetically modified cell line is deficient for a heparan sulfate catabolic enzyme.

5. The method of any one of claims 1 to 4, wherein the method comprises degranulating the cell line.

6. The method of claim 5, wherein degranulating the cell line comprises a method selected from the group consisting of Antigen-IgE induced FcεRI aggregation on the degranulating cell surface, contacting the cell line to a degranulating agent, altering the culture temperature, altering the culture medium pH, altering the culture medium salt concentration, and agitation.

7. The method of claim 6, wherein the degranulating agent is selected from the group consisting of calcium ionophore A23187, compound 48/80, tetradecanoyl phorbol acetate (TPA), and substance P.

8. The method of any one of claims 1 to 7, wherein the genetically modified cell line is cultured in CDM4NS0 medium.

9. The method of any one of claims 1 to 8, wherein the genetically modified cell line is cultured in a medium supplemented with xylosides.

10. The method of any one of claims 1 to 9, wherein the genetically modified cell line is cultured in suspension culture or in a hollow fiber bioreactor.

11. The method of any one of claims 1 to 10, wherein the genetically modified cell line overexpresses Hs3st1, Hs6st1, and Sulf-2 and is deficient for Hs2st.

12. A method of reducing blood clots in a subject, the method comprising administering a composition comprising a heparin or a heparan sulfate produced according to the method of any one of claims 1 to 11.

13. A cell comprising a genetically modified mastocytoma cell that overexpresses one or more of heparan sulfate-glucosamine 3-sulfotransferae 1 (Hs3st1), heparan sulfate 6-O-sulfotransferase 1 (Hs6st1), heparan sulfate 6-O-sulfotransferase 2 (Hs6st2), N-deacetylase and N-sulfotransferase 2 (Ndst2), and Sulf-2.

14. The cell of claim 13, wherein the cell is deficient for one or more of chondroitin sulfate N-acetylgalactosaminyltransferase 1 (Csgalnact1), chondroitin sulfate N-acetylgalactosaminyltransferase 2 (Csgalnact2), chondroitin sulfate synthase 1 (Chsy1), and Heparan sulphate 2-O-sulphotransferase (Hs2st).

15. The cell of claim 13 or claim 14, wherein the mastocytoma cell is selected from the group consisting of MST cells, P815 cells, MC/9 cells, SI/SI4 cells, 10P2 cells, 11P0-1 cells, and 10P12 cells.

16. The cell of any one of claims 13 to 15, wherein the cell is deficient for a heparan sulfate catabolic enzyme.

17. The cell of any one of claims 13 to 16, wherein the cell overexpresses Hs3st1, Hs6st1, and Sulf-2 and is deficient for Hs2st.
